(19) United States
(12) Patent Application Publication
(10) Pub. No.: US 2025/0387068 A1
(43) Pub. Date: Dec. 25, 2025

(54) APPARATUS AND METHOD FOR MEASURING AND APPLYING HIGH VOLTAGE THROUGH A MATRIX OF WIRES AND CONDUCTIVE ELEMENTS

(71) Applicant: CardioNXT, Inc., Westminster, CO (US)

(72) Inventors: Paul KESSMAN, Lakewood, CO (US); Keith MARTIN, Superior, CO (US); Jerome Ranjeev EDWARDS, Erie, CO (US); Ashwin MATHUR, Thornton, CO (US); Christian EDWARDS, Erie, CO (US)

(21) Appl. No.: 19/305,298

(22) Filed: Aug. 20, 2025

Related U.S. Application Data
(63) Continuation-in-part of application No. PCT/US2024/052687, filed on Oct. 23, 2024.
(60) Provisional application No. 63/592,854, filed on Oct. 24, 2023.

(51) Int. Cl. A61B 5/367; A61B 18/00; A61B 18/14; A61B 18/16

(52) U.S. Cl. CPC A61B 5/367; A61B 18/1492; A61B 18/16; A61B 2018/00577

(57) ABSTRACT

Devices and methods for cardiac mapping and ablation are described. In some embodiments, an apparatus for cardiac mapping and ablation includes an elongated tube with a distal and proximal end, connected to a matrix of tubes that create a surface attached to the distal end of the first tube, with at least one conductive wire parallel to the first tube, and multiple conductive elements, electrically isolated from the conductive wire, attached to the matrix of tubes. The tube and matrix apparatus constructed of materials bio compatibly suitable for introduction into the human vascular system and heart

### CROSS-REFERENCE TO RELATED APPLICATIONS

This application is a continuation-in-part of International Patent Application No. PCT/US2024/052687, filed October 23, 2024, entitled “Apparatus and Method for Measuring and Applying High Voltage Through a Matrix of Wires and Conductive Elements,” which claims priority to and the benefit of U.S. Provisional Patent Application No. 63/592,854, filed Oct. 24, 2023, and titled “Apparatus and Method for Measuring and Applying High Voltage Through a Matrix of Wires and Conductive Elements,” the disclosures of which are hereby incorporated by reference in their entireties.

### TECHNICAL FIELD

The present disclosure relates to systems, devices, and methods for assisting physicians in performing surgical procedures on patients, and more specifically, this disclosure describes systems and methods for sensing cardiac signals of the heart of a subject and/or patient and delivering therapeutic energy to treat cardiac arrhythmias.

### BACKGROUND

Medical procedures to treat cardiovascular diseases have evolved in recent years towards less invasive techniques. Physicians can now insert a small medical device into a subject and/or patient through a small incision, navigate the device through vasculature to the heart, and deliver a specific treatment to a target site. The success of these medical procedures relies on the use of specialized tools (e.g., position sensing systems) that enable the creation of a three-dimensional (3D) geometries and/or maps of the patient anatomy, which can be used to accurately locate target region(s) for treatment and navigate a device to those target region(s) to deliver a specific treatment. Current position sensing systems include mapping catheters designed to create a three-dimensional geometry of the patient's anatomy by approximating and/or touching the catheter to in vivo tissue while gathering physiologic information about the tissue to be displayed on the 3D geometry. These mapping catheters do not have adequate capabilities to deliver treatment, and thus, once a 3D map of the patient's anatomy and physiologic condition is created, the mapping catheter is removed, and a second catheter is introduced to deliver treatment. For example, catheter ablation procedures require introducing a mapping catheter to create a 3D map of the patient's anatomy. The mapping catheter is then removed, and an ablation catheter is advanced (using the 3D map) to a target location for delivery of ablative energy to cauterize ectopic tissue causing the cardiac arrhythmia. The use of multiple catheters that need to be exchanged throughout an interventional medical procedure increases the risk and inefficiency of the procedure. Consequently, there is a need in the art for systems and devices that can incorporate both mapping capabilities and delivery of treatment.

### SUMMARY

Illustrative embodiments of the present invention that are shown in the drawings are summarized below. These and other embodiments are more fully described in the Detailed Description section. It is to be understood, however, that there is no intention to limit the invention to the forms described in this Summary of the Invention or in the Detailed Description. One skilled in the art can recognize that there are numerous modifications, equivalents, and alternative constructions that fall within the spirit of the invention as expressed in the claims

In some embodiments, an apparatus can comprise a conduit that has a proximal end, a distal end, and defines a longitudinal axis extending from the proximal end to the distal end. The apparatus further comprises a contact assembly disposed on, extending from, and/or coupled to the distal end of the conduit. The contact assembly includes a conductive wire, and a plurality of supporting tubes coupled to the distal end of the conduit. The contact wire is configured to deliver irreversible electroporation ablation to a patient. The plurality of tubes extend distally and/or away from the distal end of the conduit. The contact assembly further includes a plurality of conductive elements disposed on the plurality of supporting tubes. Each conductive element from the plurality of conductive elements is electrically isolated from the conductive wire.

In some embodiments, an apparatus includes a conduit having a proximal end and a distal end, and defines a longitudinal axis extending from the proximal end to the distal end. The apparatus further includes a contact assembly that extends from the distal end of the conduit. The contact assembly includes a monolithic connecting component. The connecting component is configured to provide structural support to the contact assembly, including a tube support section and a lateral support section that is disposed between the tube support section and a distal tip of the contact assembly. The contact assembly further includes a plurality of tubes disposed on the tube support section of the connecting component. The contact assembly further includes a plurality of conductive elements that are disposed on the plurality of tubes. The plurality of conductive elements are configured to measure physiological signals associated with the patient.

In some implementations, the plurality of tubes (or a portion of the plurality of tubes) terminate proximal to the lateral support section.

In some embodiments, an apparatus includes a conduit having a proximal end and a distal end. The conduit defines a longitudinal axis extending from the proximal end to the distal end. The apparatus further includes a contact assembly. The contact assembly extends from the distal end of the conduit. The contact assembly includes a connecting component, a plurality of tubes, a plurality of conductive elements, and an elongated conductor. The connecting component is configured to provide structural support to the contact assembly, including a tube support section and a lateral support section disposed between the tube support section and a distal tip of the contact assembly. The plurality of tubes are disposed on the tube support section of the connecting component. The plurality of conductive elements are disposed on the plurality of tubes and are configured to measure physiological signals associated with a patient. The elongated conductor is coupled to the connecting component and is configured to deliver electroporation ablation therapy to the patient. Each conductive element from the plurality of conductive elements is electrically isolated from the elongated conductor.

In some implementations, the plurality of tubes (or a portion thereof) terminate proximal to the lateral support section.

In some embodiments, the plurality of conductive elements is configured to measure physiological signals from various locations on tissue of a heart of a user.

In some embodiments, the plurality of conductive elements is configured to measure the physiological signals to collectively construct a three-dimensional map of the heart of the user.

In some embodiments, the conductive elements are ring electrodes, each ring electrode having a nominal length of at least about 0.5 mm to no more than about 1.0 mm.

In some embodiments, each ring electrode has a nominal outer diameter of at least about 0.6 mm to no more than about 0.85 mm.

In some embodiments, one or more of the conductive elements from the plurality of conductive elements are configured to measure a voltage with respect to a ground electrode, the ground electrode being spaced from the plurality of conductive elements, the voltage being associated with depolarization of the tissue of the heart during a heartbeat.

In some embodiments, the plurality of conductive elements is configured to measure a plurality of voltages to collectively construct a three-dimensional map of electrical propagation through the heart during a heartbeat.

In some embodiments, the plurality of supporting tubes include structurally weak points such that the plurality of supporting tubes can be flexed concavely or convexly

In some embodiments the apparatus further comprises a connecting component coupled to a distal end of each supporting tube from the plurality of supporting tubes, the connecting component configured to place the contact assembly in an extended configuration in which the plurality of supporting tubes is oriented along a plane and each supporting tube from the plurality of supporting tubes is disposed parallel to all the other supporting tubes from the plurality of supporting tubes such that the contact assembly can lay flat at a close proximity of intracardiac tissue of a patient.

In some embodiments, the contact assembly is configured to transition from the extended configuration to a compressed configuration in which the plurality of supporting tubes are compressed towards each other such that the contact assembly has an outer diameter substantially similar to an outer diameter of the conduit.

In some embodiments, the outer diameter of the conduit is no more than about 8.5 Fr.

In some embodiments, the conductive wire is configured to establish a potential with respect to one or more conductive elements from the plurality of conducting elements, the potential capable of delivering energy to cause irreversible electroporation of cardiac cells included in tissue of a heart of a patient.

In some embodiments, the potential is at least about 1000 volts.

In some embodiments, the conductive wire is made of nitinol.

In some embodiments, the conductive wire has a nominal diameter of at least about 0.2 millimeters and no more than about 1 mm.

In some embodiments, the conductive wire has a nominal length of at least 2 mm and no more than about 20 mm.

In some embodiments, a portion of the conductive wire is disposed within the conduit, the portion of the conductive wire being surrounded by an insulating layer configured to insulate the conductive wire from the plurality of conductive elements.

In some embodiments, the insulating layer includes a polyimide material.

In some embodiments, an apparatus can comprise a conduit having a proximal end and a distal end. The conduit can define a longitudinal axis extending from the proximal end to the distal end. The apparatus can further comprise an electromagnetic localization sensor; and a contact assembly coupled to and extending from the distal end of the conduit. The contact assembly can include a conductive wire oriented parallel to the longitudinal axis; a plurality of supporting tubes, and a plurality of conductive elements. The conductive wire can be configured to deliver irreversible electroporation ablation therapy to a patient. The plurality of supporting tubes can be coupled to and extend away from the distal end of the conduit and include copper wound coils. The copper wound coils are configured to be operably coupled to the electromagnetic localization sensor to determine a location of the apparatus within an anatomy of the patient. The plurality of conductive elements can be disposed on the plurality of supporting tubes; with each conductive element from the plurality of conductive elements electrically being isolated from the conductive wire.

In some embodiments, the outer diameter of the conduit is no more than about 8.5 Fr.

In some embodiments, the conductive wire is configured to establish a potential with respect to one or more conductive elements from the plurality of conducting elements, the potential capable of delivering energy to cause irreversible electroporation of cardiac cells included in tissue of a heart of a patient.

In some embodiments, the potential is at least about 1000 volts.

In some embodiments, the conductive wire is made of nitinol.

In some embodiments, the conductive wire has a nominal diameter of at least about 0.2 millimeters and no more than about 1 mm.

In some embodiments, the conductive wire has a nominal length of at least 2 mm and no more than about 20 mm.

In some embodiments, a portion of the conductive wire is disposed within the conduit, the portion of the conductive wire being surrounded by an insulating layer configured to insulate the conductive wire from the plurality of conductive elements.

In some embodiments, wherein the insulating layer includes a polyimide material.

In some embodiments an apparatus can comprise: a conduit having a proximal end and a distal end, the conduit defining a longitudinal axis extending from the proximal end to the distal end; an electromagnetic localization sensor; and a contact assembly disposed on the distal end of the conduit. The contact assembly can include: a conductive wire oriented perpendicular to the longitudinal axis; a plurality of supporting tubes coupled to and extending away from the distal end of the conduit; and a plurality of conductive elements disposed on the plurality of supporting tubes; each conductive element from the plurality of conductive elements electrically isolated from the conductive wire.

In some embodiments, the conductive wire is configured to establish a potential with respect to one or more conductive elements from the plurality of conducting elements, the potential capable of delivering energy to cause irreversible electroporation of cardiac cells included in tissue of the heart of the user.

In some embodiments, the potential is at least about 1000 volts.

In some embodiments, the conductive wire is made of nitinol.

In some embodiments, the conductive wire has a nominal diameter of at least about 0.2 millimeters and no more than about 1 mm.

In some embodiments, the conductive wire has a nominal length of at least 2 mm and no more than about 20 mm.

In some embodiments, a portion of the conductive wire is disposed within the conduit, the portion of the conductive wire being surrounded by an insulating layer configured to insulate the conductive wire from the plurality of conductive elements.

In some embodiments, the insulating layer includes a polyimide material.

### BRIEF DESCRIPTION OF THE DRAWINGS

FIG. 1 shows a schematic illustration of an intracardiac apparatus for mapping the anatomy of a patient and delivering ablation therapy (e.g., irreversible and/or reversible electroporation), according to an embodiment of the present disclosure.

FIG. 2 illustrates a top view of an intracardiac apparatus for mapping the anatomy of a patient and delivering ablation therapy (e.g., irreversible and/or reversible electroporation), according to an embodiment of the present disclosure.

FIG. 3 illustrates a top view of an intracardiac apparatus for mapping the anatomy of a patient and delivering ablation therapy (e.g., irreversible and/or reversible electroporation), according to an embodiment of the present disclosure.

FIG. 4 illustrates a side view of the intracardiac apparatus shown in FIG. 3

FIG. 5 illustrates a perspective view of the intracardiac apparatus shown in FIG. 3.

FIG. 6 illustrates a cross-sectional front view of a conduit of an intracardiac apparatus displaying electrical connections for conductive elements and conductive wires, according to an embodiment of the present disclosure.

FIGS. 7A and 7B illustrate connectors disposed at a distal end of a conduit, displaying spacings for insulating multiple conductive elements of an intracardiac device, according to embodiments of the present disclosure.

FIG. 8 illustrates a cross-sectional front view of a conduit of an intracardiac apparatus displaying multiple channels for (i) accommodating electrical connections for conducting elements and conductive wires, (ii) flowing irrigation solutions, and (iii) housing electromagnetic sensors and deflection elements, according to an embodiment of the present disclosure

FIG. 9 illustrates a handle of an intracardiac apparatus (or coupled to the intracardiac apparatus) including a dial for deflecting conductive elements and conductive wires, and a cable output connector configured to be removably coupled to the handle, according to an embodiment of the present disclosure

FIGS. 10A and 10B illustrate a side view and a top view respectively, of two interconnected printed circuit boards (PCBs) displaying the electrical connection of the handle and the output cable connector shown in FIG. 9.

FIG. 11 illustrates a perspective view of the two interconnected printed circuit boards (PCBs) shown in FIGS. 10A and 10B.

FIG. 12A illustrates a cross-sectional side view of the handle shown in FIG. 9.

FIG. 12B illustrates a top view of the PCB of the handle shown in FIGS. 9 and 12A.

FIG. 13 illustrates a perspective view of an intracardiac apparatus for mapping the anatomy of a patient and delivering ablation therapy (e.g., irreversible and/or reversible electroporation), according to an embodiment of the present disclosure.

FIGS. 14A and 14B illustrate a perspective view and a top view, respectively, of a connecting component included in the intracardiac apparatus shown in FIG. 13.

FIGS. 15A and 15B illustrates a perspective view and a top view, respectively, of an intracardiac apparatus for mapping the anatomy of a patient, according to an embodiment of the present disclosure.

FIGS. 16A, 16B, and 16C illustrate a perspective view of the intracardiac apparatus of FIGS. 15A and 15B during transition of the contact assembly from an expanded configuration to a compressed configuration.

FIG. 17 illustrates a method for measuring physiological signals and delivering ablation therapy to tissue of a heart of a patient, according to an embodiment of the present disclosure.

### DETAILED DESCRIPTION

Catheters have increasingly become the preferred approach and/or method to diagnose and treat cardiovascular diseases. Catheters can be inserted via small incisions in the body of a subject and/or patient and advanced to specific locations in the body of the patient which are otherwise inaccessible without the use of a more invasive procedure. Catheters can be used for diagnosing a disease and/or for delivering a treatment to a target region in the body of the patient. For example, catheter ablation procedures can be used to diagnose and treat cardiac arrhythmias. This type of procedure typically requires introducing a first catheter (e.g., a mapping catheter) to create a three-dimensional (3D) geometry (e.g., a map) of the patient's heart by approximating and/or touching the catheter to in vivo tissue while gathering physiologic information about the tissue to be displayed on the 3D geometry. Once the 3D map of the patient's heart has been created with the mapping catheter, a second catheter (e.g., an ablation catheter) can be inserted and navigated with the aid of the 3D map generated by the mapping catheter, to a target treatment location and/or region. The ablation catheter can then deliver a therapy to tissue disposed on the target treatment location and/or region, typically in the form of thermal ablation. In some instances, after delivering the therapy with the thermal ablation catheter, the mapping electrode is reintroduced to remap the patient's hearth and confirm adequate treatment. The exchange of catheters during the intervention provides opportunities for complications and errors, and thus considerably increases the risk and inefficiency of the medical procedure.

The need for a mapping catheter and separate ablation catheter for the treatment of cardiac arrhythmias stems from the different characteristics, geometries, and/or configurations needed for each catheter in order to achieve optimal operation. Mapping catheters include an array of small size electrodes, often referred to as microelectrodes, disposed, attached, and/or integrated along the longitudinal axis of the catheter. Mapping microelectrodes can be annular structures (e.g., rings) made of platinum or other metals, having relatively small dimensions such as for example, 1 mm long×0.8 mm diameter. During operation, the array of microelectrodes are oriented flat on in vivo tissue to measure signals of the tissue with high fidelity, minimizing noise caused by signals produced in other areas of the heart. Unlike mapping catheters, thermal ablation catheters use much larger electrodes compared to the mapping microelectrodes. Thermal ablation involves heating or freezing the tissue to the point of necrosis. To achieve this, thermal ablation catheters deliver energy in the form of a high current density discharge produced with large size electrodes attached and/or integrated into the catheter. The need for small outer diameters for mapping catheters (e.g., outer diameter <8.5 French gauge, Fr) and considerably larger outer diameters therapeutic ablation catheters (e.g., outer diameters >8.5 Fr) has precluded the successful integration of mapping and ablation capabilities into a single catheter, particularly for catheters requiring high current densities for treating large treatment location and/or region.

The present disclosure provides devices and methods for generating three-dimensional geometries of the anatomy of a patient (e.g., mapping the anatomy of the patient) and delivering therapeutic ablation energy to localized areas of interest. In some embodiments, the devices and methods described herein utilize a non-thermal ablative modality, known as irreversible electroporation ablation or pulse field ablation (PFA) for the treatment of cardiac arrythmias. Alternatively, and/or additionally, in some embodiments the devices and methods disclosed herein use reversible electroporation for delivering drugs to specific and/or desired locations. Irreversible electroporation ablation involves applying high voltage pulses between two electrodes that results in destabilization of the cellular membrane and formation of pores inducing cell tissue death. Reversible electroporation applies the high voltage pulses between two electrodes to induce the destabilization of the cellular membrane and formation of pores to introduce one or more drugs, medicaments, therapies, etc., to a target location within the cells (e.g., drug delivery). After drug delivery the pores are allowed to shrink and/or close. In some implementations, reversible electroporation can be used to target particular cells to open for drug delivery, and one or more drugs can be delivered to a region near or surrounding those particular cells, such that the one or more drugs will be absorbed only (or substantially only) by the cells opened by the reversible electroporation, and e.g., not absorbed by other or surrounding cells separate from the target cells. Reversible/irreversible electroporation is a technique that is much less dependent on creating high current densities on the tissue often used in thermal ablation methods. The present disclosure provides intracardiac catheters that incorporate electrodes and electrode configurations that are optimal for mapping as well delivery of irreversible (and/or reversible) electroporation while preserving the form factor of diagnostic catheters. Consequently, the devices described herein provide physicians the ability to map with high fidelity while also delivering ablation therapy (e.g., irreversible electroporation or reversible electroporation) in diagnostic and therapeutic cardiac arrhythmia procedures. While the devices and methods described herein are intended to be used in the treatment of heart tissue, the devices and methods could be introduced into other anatomies for mapping and ablation of various tissues. One such example could be the introduction of a catheter similar to the apparatus described herein through the urethra to a region adjacent to the prostate capsule, followed by electroporation ablation to either kill cancer or debulk the size of a prostate in cancer and benign prostate hyperplasia patients. In some implementations the catheter could also be used for mapping the urethra, prostate, and/or surrounding anatomy. Other examples include mapping and/or ablation within the esophagus, renal artery, and/or any other locations suitable for mapping and/or ablation.

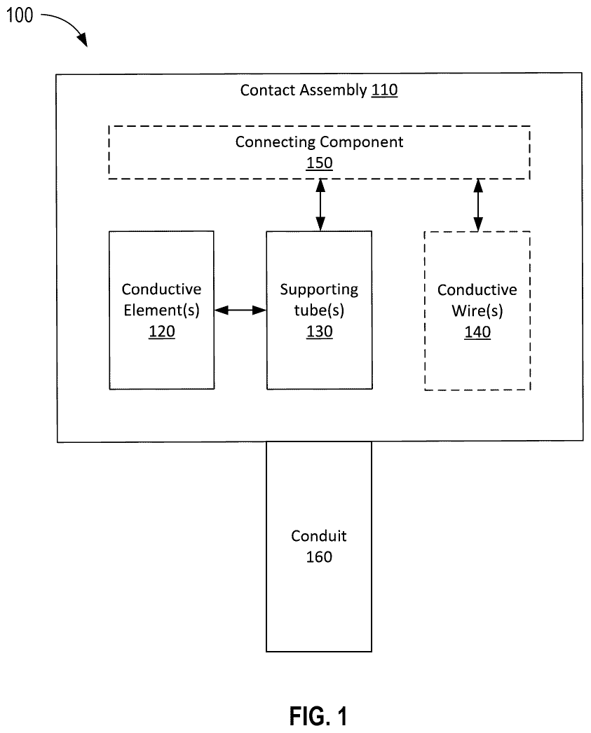

Now referring to the drawings, FIG. 1 shows a schematic illustration of an intracardiac apparatus 100 for mapping the anatomy of a patient and delivering ablation therapy (e.g., irreversible electroporation or pulse field ablation, PFA, and/or reversible electroporation), according to an embodiment of the present disclosure. The intracardiac apparatus 100, which can also be referred to as the apparatus 100, or the catheter 100, includes a contact assembly 110 disposed on, extending from, and/or coupled to, a distal end of a conduit 160. The contact assembly 110 can include a plurality of conductive element(s) 120, and a plurality of supporting tube(s) 130. Optionally (as illustrated by broken lines), in some embodiments the contact assembly 110 can also include one or more conductive wire(s) 140 and a connecting component 150. The contact assembly 110 is configured to assume a first configuration, also referred to as a compressed or delivery configuration, in which the contact assembly 110 and/or its components are collectively collapsed and/or constrained along a longitudinal axis defined by the conduit 160 and at a short distance from each other, such that a cross-sectional area of the contact assembly 110 is substantially similar to and/or the same as the outer diameter of the conduit 160. For example, in some embodiments, the contact assembly 110 can assume a compressed configuration in which all the components included in the contact assembly 110 are aligned along the longitudinal axis of the conduit 160 resulting in an outer diameter of no more than about 8.5 Fr. This outer diameter of the contact assembly 110 in the compressed configuration is comparable and/or similar to the outer diameter of many diagnostic catheters. In the compressed configuration, the apparatus 100 can be advanced through the vasculature of the heart of a patient to reach a specific treatment location and/or region. The contact assembly 110 can be transitioned from the compressed configuration to a second, expanded, unconstrained configuration such that the contact assembly 110 can then be used for mapping and/or ablation. In the expanded configuration the components of the contact assembly 110 can be arranged in any orientation suitable to measure and/or capture signals of the tissue (e.g., for mapping and/or diagnostics) and deliver ablation energy. In some implementations, in the expanded configuration, components of the contact assembly 110 are oriented parallel to each other and along a plane such that they can be brought into close proximity and/or direct contact with in vivo tissue of the specific treatment location and/or region. Said in other words, in the expanded configuration the components of the contact assembly 110 can be oriented to lay flat at a very close proximity or in direct contact with the tissue of the specific treatment location and/or region. In the expanded configuration components of the contact assembly 110 such as the conductive element(s) 120 can be used to measure and/or capture signals of the tissue with high fidelity, which can then facilitate the generation of a 3D map of the anatomy of the patient such as for example, the heart of the patient or region thereof, as further described herein. In the expanded configuration, the conductive wire(s) 140 can also be used to emit voltages that deliver ablation therapy such as irreversible electroporation therapy to the target location and/or region. The components of the contact assembly 110 are designed to naturally transition between the compressed configuration and the expanded configuration to facilitate disposing the apparatus 100 within a delivery catheter or a delivery sheath which can be navigated through the anatomy of the patient to position the apparatus 100 at a target treatment location and/or region. For example, the contact assembly 110 can assume the compressed configuration when the apparatus 100 is introduced within a delivery catheter and/or delivery sheath (not shown) and be then introduced via an incision into the body of a patient. The contact assembly 110 can then transition from the compressed configuration to the expanded configuration to facilitate mapping and/or delivering irreversible (and/or reversible) electroporation therapy on target regions. In some implementations, the apparatus 100 can be configured to conduct intracardiac mapping and/or ablation by disposing the apparatus 100 at endocardial target regions (e.g., regions disposed within the heart of a patient). In some implementations, the apparatus 100 can be configured to conduct mapping and/or ablation by disposing the apparatus 100 on epicardial target regions (e.g., regions disposed outside and/or on the surface of the heart of a patient). In such embodiments, in some instances, the contact assembly 110 of the apparatus 100 can be introduced into the pericardial space of the heart of the patient with a sub subxiphoid percutaneous access point. (e.g., poke a small in the center of a patient's chest). In some cases, epicardial tissue causing arrhythmia is inaccessible from inside the heart, and so the apparatus 100 in such instances can map and/or ablate the epicardial tissue from outside the heart.

The conductive element(s) 120 can be a plurality of electrodes disposed on or otherwise coupled to the supporting tube(s) 130 configured to contact tissue of the patient to facilitate high fidelity signal acquisition, e.g., to precisely identify cellular ectopic foci. In some embodiments the conductive element(s) 120 can be used to measure electrical data such as voltage, current, impedance, and/or depolarization. The voltages measured by the conductive element(s) 120 can be used to assess the health of the tissue. Low voltage measurements can be used to identify areas of scar tissue while higher voltage measurements can be used to identify areas of healthy tissue that is depolarizing with each heartbeat. In some embodiments, the electrical data measured by the conductive element(s) 120 can be associated with three dimensional locations in space that facilitate the reconstruction of 3D physiologic maps of certain anatomy of a patient such as the heart. For example, in some embodiments one or more conductive element(s) 120 can be disposed on and/or otherwise coupled to the conduit 160, to be used as a local ground to facilitate taking measurements from the conductive element(s) 120 disposed on the supporting tube(s) 130 of the contact assembly 110. The conductive element(s) 120 can collect localization data using an impedance-based localization system, facilitating correlating the physiological data measured by the conductive element(s) 120 with their relative position within the heart of the patient, thus generating a 3D map of the heart. Voltage or scar data measured by the conductive element(s) 120 can be displayed on a 3D model and/or map of the heart. Depolarization measurements gathered by the conductive element(s) 120 enable the mapping of electrical propagation through the heart. Additionally or alternatively, in some embodiments, the contact assembly 110 can include copper wound coils disposed within and/or about the supporting tube(s) 130 which can be used with electromagnetic sensors (e.g., disposed on the conduit 160 or contact assembly 110) to determine the relative position of the contact assembly 110 or a portion thereof. These 3D maps can be used for diagnosis purposes such as identifying and/or determining clinical issue such as a cardiac arrhythmia. The 3D maps can also be used for navigation purposes such as to guide the delivery of a therapy without the use of harmful ionizing radiation techniques such as X-Rays. Furthermore, the 3D maps developed with the aid to the conductive element(s) 120 included in the contact assembly 110 can be used as confirmatory post therapy delivery maps to assess if a particular therapy was delivered in the intended manner, the intended region, and whether further therapy is needed.

In some embodiments, the conductive element(s) 120 can be electrodes having an annular shape mounted on an external surface of the supporting tube(s) 130. In some such embodiments the conductive element(s) 120 can be ring electrodes disposed along the external diameter of the supporting tube(s) 130. In some embodiments the conductive element(s) 120 can be ring electrodes having a nominal outside diameter (OD) of at least about 0.50 mm, at least about 0.55 mm, at least about 0.60 mm, at least about 0.65 mm, at least about 0.70 mm, at least about 0.75 mm, at least about 0.80 mm, at least about 0.85 mm, at least about 0.90 mm, at least about 0.95 mm, at least about 1.0 mm, at least about 1.1 mm, at least about 1.2 mm, at least about 1.3 mm, at least about 1.4 mm, or at least about 1.5 mm, inclusive of all ranges and values therebetween. In some embodiments, the conductive element(s) 120 can be ring electrodes having a nominal OD of no more than about 1.5 mm, no more than about 1.45 mm, no more than about 1.35 mm, no more than about 1.25 mm, no more than about 1.05 mm, no more than about 0.95 mm, no more than about 0.85 mm, no more than about 0.75 mm, no more than about 0.65 mm, or no more than about 0.50 mm, inclusive of all ranges and values therebetween.

Combinations of the above referenced ranges for the nominal OD of the ring electrodes are also possible (e.g., a nominal OD of at least about 0.6 mm to no more than about 0.85 mm, or at least about 0.6 mm to more than about 1.5 mm).

In some embodiments, the conductive element(s) 120 can be ring electrodes having a nominal length of at least about 0.5 mm, at least about 0.6 mm, at least about 0.7 mm, at least about 0.7 mm, at least about 0.8 mm, at least about 0.9 mm, at least about 1.0 mm, at least about 1.1 mm, at least about 1.2 mm, at least about 1.3 mm, at least about 1.4 mm, at least about 1.5 mm, at least about 1.6 mm, at least about 1.7 mm, at least about 1.8 mm, at least about 1.9 mm, or at least about 2.0 mm, inclusive of all ranges and values therebetween. In some embodiments, the conductive element(s) 120 can be ring electrodes having a nominal length of no more than about 2.0 mm, no more than about 1.8 mm, no more than about 1.6 mm, no more than about 1.4 mm, no more than about 1.2 mm, no more than about 1.0 mm, no more than about 0.95 mm, no more than about 0.85 mm, no more than about 0.75 mm, no more than about 0.65 mm, no more than about 0.55 mm, no more than about 0.45 mm, or no more than about 0.40 mm, inclusive of all ranges and values therebetween.

Combinations of the above referenced ranges for the nominal length of the ring electrodes are also possible (e.g., a length of at least about 0.5 mm to no more than about 1.0 mm, or at least about 0.5 mm to more than about 2.0 mm).

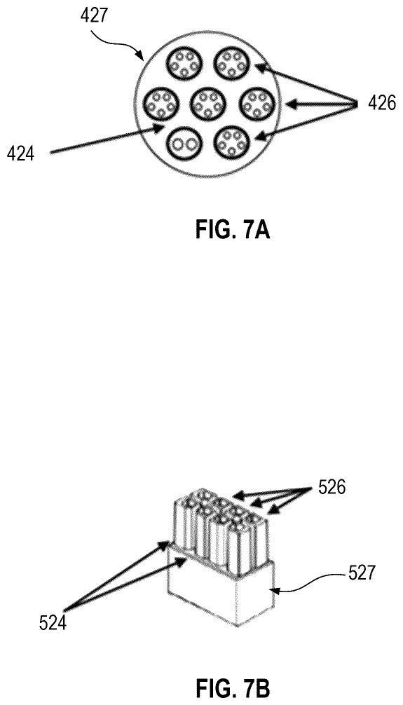

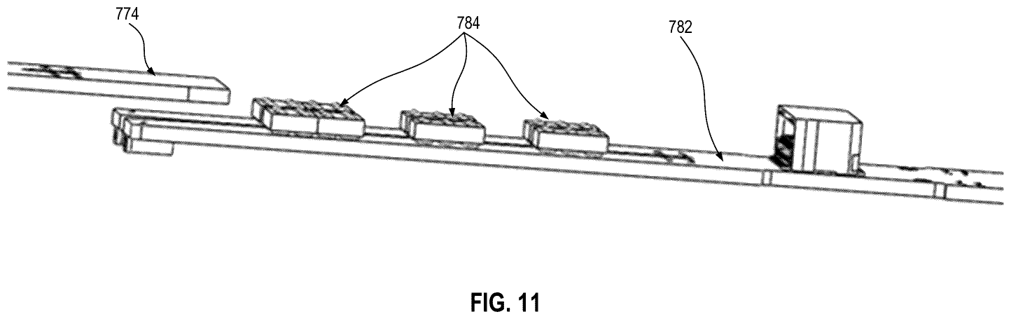

The conductive element(s) 120 can be made of metals such as platinum, or platinum alloys. Each conductive element 120 can include a lead wire coupled to and/or attached to the conductive element 120 to connect electrically the conductive element 120 with a connector similar to and/or the same as the connectors shown in FIGS. 7A, 7B, and 11. These connectors can be electrically and operably coupled the conductive element(s) 120 to a cardiac mapping system, a pulse field ablation (PFA) generator, or any other suitable device capable of generating 3D maps of the anatomy of a patient and/or delivering energy therapies. For example, in some embodiments, the conductive element(s) 120 can include lead wires configured to connect the surface of the conductive element(s) 120 which are at close proximity or in direct contact with intracardiac tissue of a patient, to an external device such as an electrocardiogram recording system (EKG) configured to measure physiological parameters of the heart of the patient. The lead wires can be made of a conductive metal such as copper, silver, nickel, aluminum, or alloys thereof. Each lead wire can be routed through a lumen, interior volume, and/or cavity of the supporting tube(s) 130 from a point of contact and/or connection of the lead wire with the conductive element 120, to a distal end of the conduit 160. Each lead wire can then be routed through a shaft and/or interior volume of the conduit 160 from the distal end (or distal end-portion) of the conduit 160 to a proximal end (or proximal end-portion) of the conduit 160, where the lead wire can be coupled to a connector (not shown in FIG. 1.). In use, the conductive element(s) 120 can be used to deliver electrical signals (voltages and/or currents) from the source, and via the lead wires, to intracardiac tissue of a patient in direct contact and/or in close proximity of the conductive element(s) 120. The conductive element(s) 120 can be used to measure physiologic parameters of the intracardiac tissue of the patient such as voltage and/or timing of cardiac depolarization. In some embodiments, each conductive element 120 from the plurality of conductive element(s) 120 can be a ring electrode having its own lead wire that connects the ring electrode with a power source and/or a meter. Furthermore, in some embodiments, each lead wire can be electrically insulated from other lead wires connected to other ring electrodes and/or conductive element(s) 120.

In some embodiments, the conductive element(s) 120 can be ring electrodes having lead wires of predetermined dimensions. For example, in some embodiments a lead wire of a ring electrode can have a nominal diameter of at least about 36 American Wire Gauge (AWG), at least about 37 AWG, at least about 38 AWG, at least about 39 AWG, at least about 40 AWG, at least about 41 AWG, at least about 42 AWG, at least about 43 AWG, at least about 44 AWG, at least about 45 AWG, at least about 46 AWG, at least about 47 AWG, or at least about 48 AWG, inclusive of all ranges and values therebetween. In some embodiments, a lead wire of a ring electrode can have a nominal diameter of no more than about 48 AWG, no more than about 46 AWG, no more than about 44 AWG, no more than about 42 AWG, no more than about 40 AWG, no more than about 38 AWG, or no more than about 36 AWG, inclusive of all ranges and values therebetween.

Combinations of the above referenced ranges for the nominal diameter of the lead wires of the ring electrodes are also possible (e.g., a nominal diameter of at least about 38 AWG to no more than about 42 AWG, or at least about 36 AWG to more than about 48 AWG.).

In some embodiments, the lead wires connecting the conductive element(s) 120 to an external device can be insulated to prevent short circuiting and/or arching of the conductive element(s) 120 with other conductive element(s) 120 or with the one or more conductive wire(s) 140. It is worth noticing that the need to provide robust insulation for the conductive element(s) 120 and the conductive wire(s) 140 imposes a significant challenge to the integration of a catheter that integrates irreversible (and/or reversible) electroporation ablation capabilities with mapping capabilities. The large voltages used during irreversible (and/or reversible) electroporation ablation can lead to short circuiting and arching of the lead wires connected to the conductive element(s) 120 and the conductive wire(s) 140, which can affect the stability, reproducibility and overall accuracy of the mapping information produced by the catheter. The apparatus and systems described herein overcome the current limitations by introducing systems and components that enable the use of conductive wires to deliver energy therapies without impacting the mapping capabilities of the apparatus. For example, in some embodiments, the lead wires of each conductive element 120 can be protected by a layer and/or coating of an electrical insulation material that covers the external surface of the lead wire from a contact point on the conductive element 120 to a distal end of the conduit 160. The lead wires can then continue to be protected by a layer and/or coating of an electrical insulation material from the distal end of the conduit 160 to a proximal end of the conduit 160 where the lead wire is coupled to a connector, as described above. Furthermore, in some embodiments, the lead wires of the conductive element(s) 120 can be collectively insulated to prevent short circuiting the conductive element(s) 120 with the one or more conductive wire(s) 140. In such embodiments, the lead wires of the conductive element(s) 120 with their individual insulation coatings and/or layers can be collectively routed through an insulating tube such as a polyimide insulating tube to provide additional insulation from the conductive wires 140. In some embodiments, a spacing is provided between the lead wires (and their associated insulation) of the conductive element(s) 120 and the conductive wire(s) 140 travelling through the conduit 160 from a distal end of the conduit 160 to a proximal end of the conduit 160. This spacing is designed to provide additional clearance that prevents arcing between the conductive element(s) 120 and the conductive wire(s) 140 when high voltage is applied to conductive wire(s) 140, as further described herein.

In some embodiments, the conducive element(s) 120 can be disposed on the supporting tube(s) 130 forming a two-dimensional flat array and/or matrix in which the conductive element(s) 120 are organized in columns and rows at specific and/or predetermined distances from each other, as further described herein. For example, in some embodiments, the conductive element(s) 120 can be disposed on a plurality of supporting tube(s) 130 forming a 4×5 matrix. The conductive element(s) 120 can be attached, mounted, and/or integrated on 4 different supporting tube(s) 130, with each supporting tube 130 including 5 conductive element(s) 120 aligned along a first direction consecutively (e.g., disposed on the supporting tube 130 one after the other at a fixed distance and/or separation). The conductive elements 120 disposed on different supporting tubes 130 can also be oriented with respect to a second direction, perpendicular to the first direction such that adjacent conductive element(s) 120 are disposed at a fixed distance or separation from each other. Orienting the conductive element(s) 120 in a flat orientation with respect to each other and at specific distances provides a unique benefit of being able to lay flat on in vivo tissue to measure signals of the tissue with high fidelity without capturing signals from other areas of the field such as the blood pool within a heart chamber or the opposite side of the heart, which all contribute to far field noise pollution of the map being created. Orienting the conductive element(s) 120 in a known matrix orientation also allows redundant measurements to be taken from one conductive element (120) to another conductive element 120 allowing error detection and the highest fidelity of physiologic measurement of signals.

The supporting tube(s) 130 can be and/or include a plurality of structures providing an external surface that can be used to couple, mount, attach, and or integrate a plurality of conductive element(s) 120, and an interion lumen, volume, passage, cavity, and/or shaft that can accommodate various subcomponents of the contact assembly 110 such as lead wires connecting each conductive element 120 to a connector disposed on a proximal end of the conduit 160 (not shown in FIG. 1). In some embodiments the supporting tube(s) 130 can define an interior lumen, volume, passage, cavity, and/or shaft sized and configured to accommodate other components of the contact assembly 110 such as copper wound coils. As described above, the copper wound coils can be used with one or more electromagnetic position sensors to determine the position of the contact assembly 110 (or a portion thereof) with respect to the heart of a patient. In some embodiments, the interior lumen, volume, passage, cavity of the supporting tube(s) 130 can accommodate fiber optic elements. The fiber optic elements can be routed via the conduit 160 and into the supporting tube(s) 130 to illuminate and/or capture images of the surrounding anatomy (e.g., inside and/or around the heart). In some embodiments, the interior lumen, volume, passage, cavity of the supporting tube(s) 130 can accommodate an energy storage device such as a spring or the like embedded in each supporting tube 130 from the plurality of supporting tubes(s) 130. The springs can be used to provide additional flexibility to the supporting tube(s) 130 as the apparatus 100 is navigated through vasculature of the patient. In some embodiments, the interior lumen, volume, passage, cavity of the supporting tube(s) 130 can accommodate one or more thermocouples that enable measuring a temperature of the heart and/or the blood being transported therein. In some embodiments, the interior lumen, volume, passage, cavity of the supporting tube(s) 130 can accommodate at least one or more ultrasound transducer(s) embedded into one or more of the supporting tube(s) 130. The ultrasound transducers can be disposed within one or more of the supporting tube(s) 130 to generate images of the patient's anatomy, e.g., the heart, create a geometry of the heart by ranging interior regions of the heart to the heart wall, and deliver therapies such as, for example. High Intensity Focused Ultrasound (HIFU), or Extracorporeal Shock Wave Therapy (ECWT). In some embodiments, the interior lumen, volume, passage, cavity of the supporting tube(s) 130 can accommodate at least one video camera and/or sensor embedded into one or more of the supporting tube(s) 130.

The supporting tube(s) 130 can be made of a flexible material that is able to deform to accommodate small changes in the topography of the vasculature of the patient. For example, in some embodiments, one or more of the supporting tube(s) 130 can include a first portion and/or segment made of a relatively strong material (e.g., a material having mechanical high strength, hardness, impact resistances and/or fracture toughness) and at least one second portion and/or segment made of a material having relatively weaker mechanical properties with respect to the material in the first portion. The portions and/or sections having weaker mechanical properties can enable deforming the supporting tubes 130 to facilitate flexing the supporting tubes 130 concavely or convexly as needed. In some implementations, one or more portions of the supporting tubes and/or the contact assembly 110 can flex and/or assume a different orientation in response to contact with the anatomy. In some embodiments, the supporting tube(s) 130 can be made of flexible materials that enable disposing the contact assembly 110 in the expanded configuration at close proximity and/or in direct contact with tissue of the heart of the patient. As described above, in the expanded configuration the components of the contact assembly 110 can be oriented parallel to each other and along a plane which can be brought into close proximity and/or direct contact with in vivo tissue of the heart. That is to say, in the expanded configuration the components of the contact assembly 110 can be oriented to lay flat at a very close proximity or in direct contact with tissue of the heart of the patient. In some embodiments, the supporting tube(s) 130 can be deformed individually to better adjust and/to fit to the surface contour of the tissue of the heart.

The supporting tube(s) 130 can have a viscoelastic behavior that allows exerting forces to deform the supporting tube(s) 130 in order to lay flat at close proximity or at direct contact with the surface of the tissue being measured. The supporting tube(s) 130 can then recover their original position, orientation, and/or shape once the exerted forces exerted are removed. In some embodiments, the supporting tube(s) 130 can be made from a flexible or spring-like material including polymers such as Polyether Ether Ketone (PEEK), Polypropylene (PP), Polystyrene (PS), Polycarbonate (PC), polyethylene terephthalate (PET) or the like. In some embodiments, the supporting tube(s) 130 can be made of and/or include a shape-memory metal or metal alloy materials including, for example, Nickel-Titanium (e.g., Nitinol), Copper-Aluminium-Nickel, Copper-Zinc-Aluminium, Iron-Manganese-Silicon, or the like or the like. The dimensions of each supporting tube 130 (e.g., the length and/or diameter) can be selected to impart a specific property of the supporting tube(s) 130, or at least portions thereof, including for example, a resiliency, a flexibility, a stiffness, a foldability and/or a conformability. In particular, the foldability of materials such as Nitinol facilitates transitioning the contact assembly 110 between the compressed and the expanded configuration, enabling inserting the apparatus 100 into a delivery catheter or introducer to map and/or deliver ablation therapy to a patient or removing the apparatus 100 from the body of the patient after completing a procedure. In some embodiments, the properties of the materials used to fabricate and/or construct the supporting tube(s) 130 can be varied along a length of supporting tube(s) 130 at specific locations and/or regions. For example, in some embodiments, the supporting tube(s) 130 can be weakened at specific locations and/or regions to impart a desired flexibility to the supporting tube(s) 130, allowing the supporting tube(s) 130 to reversibly deform concavely or convexly. In some embodiments, for example, the support tube(s) 130 can be a shape set to default to a preferred orientation when in the expanded configuration (e.g., not constrained within a delivery catheter).

The supporting tube(s) 130 can be disposed on a distal end-portion of the conduit 160. More specifically, the supporting tube(s) 130 can disposed on the distal end-portion of the conduit 160 extending away from the distal end-portion of the conduit 160. Said in other words, the supporting tube(s) 130 can be mounted at a distal end of the conduit 160 and extend distally from the distal end of the conduit 160. In some embodiments, when the contact assembly 110 assumes the expanded configuration, the supporting tube(s) 130 can extend away from the distal end of the conduit 160 in multiple directions (e.g., the supporting tube(s) 130 extended distally from the distal end of the conduit 160 following non-parallel directions). In some embodiments, when the contact assembly 110 assumes the expanded configuration, the supporting tube(s) 130 extend away from the distal end of the conduit 160 such that each supporting tube 130 is oriented parallel to all the other supporting tubes 130. As described above, in some embodiments when the contact assembly 110 assumes the expanded configuration, each supporting tube 130 included in the contact assembly 110 can be oriented parallel to each other and along a plane which can be brought into close proximity and/or direct contact with in vivo tissue of the heart of a patient (e.g., when the contact assembly 110 lays flat at close proximity or in direct contact with the tissue). Furthermore, in some embodiments, when the contact assembly 110 assumes the expanded configuration, each supporting tube 130 included in the contact assembly 110 can be oriented parallel to each other, and also with respect to a longitudinal axis defined by the conduit 160 from a distal end to a proximal end of the conduit 160, as further described herein.

In some embodiments, when the contact assembly 110 assumes the compressed configuration, the supporting tube(s) 130 can become collapsed and/or constrained along a longitudinal axis defined by the conduit 160 at a very close proximity from each other such that the contact assembly 110 (and all of their components) have a form factor that fits within a delivery or a diagnostic catheter or a sheath (not shown). In this manner, the contact assembly 110 and the conduit 160 can fit within and be advanced within the patient's vasculature within a delivery catheter or sheath having a suitably small nominal outer diameter, such as, for example, 10 Fr, 9 Fr, 8 Fr, 7 Fr, or the like. In some implementations, the contact assembly 110 in its compressed configuration has a cross-sectional area similar to, the same as, or different from (e.g., less than) a cross-sectional area of the conduit 160. In that way, the apparatus 100 can be disposed within a delivery catheter and/or delivery sheath and be advanced through the vasculature of a patient to a target location and/or region. As described above, in some embodiments the supporting tube(s) 130 can include springs embedded in each supporting tube 130. The springs can impart flexibility to each supporting tube 130, while in some instances, prevent the supporting tube(s) 130 from retracting into the conduit 160, as further described herein. In some embodiments the supporting tube(s) 130 can include one or more wires that can mechanically couple the supporting tube(s) 130 with a dial disposed at a handle coupled to and/or included in the apparatus 100. The dial enables a user to adjust the tension on the one or more wires to flex and/or deform the supporting tube(s) 130 along with other components of the contact assembly 110 (e.g., the conductive wire(s) 140, or the connecting components 150) to better conform to the intracardiac tissue of the subject (e.g., to deform the contact assembly 110 to lay flat on the intracardiac tissue).

In some embodiments, the supporting tube(s) 130 can be coupled to a connecting component 150. The connecting component 150 can be used to facilitate maintaining the orientation and/or the relative positions of the components of the contact assembly 110 (e.g., positions with respect to each other or with respect to the conduit 160) when the contact assembly 110 assumes the expanded configuration or the compressed configuration, as further described herein. In some embodiments, a distal end-portion of each supporting tube 130 can be coupled to one or more segments and/or portions of the connecting component 150 to facilitate orienting the supporting tube(s) 130 at their intended positions and/or locations when the contact assembly 110 transitions between the compressed configuration and the expanded configuration.

The optional conductive wire(s) 140 can be any suitable structure made of electrically conductive materials such as stainless steel, copper, gold, platinum, nickel, titanium, or nitinol. Alternatively, or additionally, in some embodiments the conductive wire(s) 140 can be made a metal coated with platinum or including a radiopaque wound platinum wire to facilitate imaging the conductive wire(s) 140 with a fluoroscope. The conductive wire(s) 140 are configured to establish a potential and/or voltage between the conductive wire(s) 140 and one or more conductive element(s) 120. These voltages enable delivering energy to cause irreversible and/or reversible electroporation of cardiac cells included in tissue of the heart of a patient. In some embodiments, the conductive wire(s) 140 are configured to deliver these voltages for a short period of time according to predetermined frequencies and/or voltage intensities. Said in other words, in some embodiments the conductive wire(s) 140 can be configured to deliver the voltages, and thus the irreversible electroporation ablation, according to any suitable pulse field ablation (PFA) methodology and/or treatment. It is worth noticing that the voltages used during the irreversible and/or reversible electroporation of cardiac cells may not require direct physical contact between the tissue and the conductive wire(s) 140. Consequently, the apparatus 100 is able to effectively deliver energy therapy to the tissue using the conductive wire(s) 140 and very small conductive elements 120. In some embodiments, the conductive wire(s) 140 are disposed and/or arranged on the contact assembly 110 such that a voltage can be applied across a larger area (when the contact assembly 110 assumes the expanded configuration) while still maintaining a form factor that is very thin and able to fit within a lumen of a standard cardiac diagnostic catheter (e.g., a lumen characterized by a nominal outer diameter of no more than about 8.5 Fr).

In some embodiments, the conductive wire(s) 140 can be and/or include any suitable elongated structure (also referred to as an elongated conductor) such as a rod, tube and/or bar. In some such embodiments, the conductive wire(s) 140 can be characterized by a nominal outer diameter (OD) of at least about 0.2 mm, at least about 0.3 mm, at least about 0.4 mm, at least about 0.5 mm, at least about 0.6 mm, at least about 0.7 mm, at least about 0.8 mm, at least about 0.9 mm, or at least about 1.0 mm, inclusive of all ranges and values therebetween. In some embodiments, the conductive wire(s) 140 can be and/or include an elongated structure such as a rod, tube, and/or bar characterized by a nominal outer diameter (OD) of no more than about 1.0 mm, no more than about 0.95 mm, no more than about 0.85 mm, no more than about 0.75 mm, no more than about 0.65 mm, no more than about 0.55 mm, no more than about 0.45 mm, no more than about 0.35 mm, or no more than about 0.2 mm, inclusive of all ranges and values therebetween. In some embodiments, the conductive wire(s) 140 can be and/or include a hollow structure defining a lumen and/or interior volume that can be used to accommodate a portion of the connecting component 150, as further disclosed herein. In such embodiments, the conductive wire(s) 140 can be characterized by a nominal outer diameter (OD) of at least about 0.2 mm, at least about 0.3 mm, at least about 0.4 mm, at least about 0.5 mm, at least about 0.6 mm, at least about 0.7 mm, at least about 0.8 mm, at least about 0.9 mm, or at least about 1.0 mm, inclusive of all ranges and values therebetween.

Combinations of the above referenced ranges for the nominal outer diameter (OD) of the conductive wire(s) 140 are also possible (e.g., a nominal OD of at least about 0.2 mm to no more than about 1.0 mm, or at least about 0.6 mm to more than about 0.85 mm).

In some embodiments, the conductive wire(s) 140 can be characterized by a nominal length of at least about 2 mm, at least about 3 mm, at least about 4 mm, at least about 6 mm, at least about 8 mm, at least about 10 mm, at least about 12 mm, at least about 14 mm, at least about 16 mm, or at least about 20 mm, inclusive of all ranges and values therebetween. In some embodiments, the conductive wire(s) 140 can be characterized by a nominal length of no more than about 20 mm, no more than about 17.5 mm, no more than about 15 mm, no more than about 13.5 mm, no more than about 10 mm, no more than about 9 mm, no more than about 8 mm, no more than about 7 mm, no more than about 6 mm, no more than about 5 mm, no more than about 4 mm, no more than about 3 mm, or no more than about 2 mm, inclusive of all ranges and values therebetween.

Combinations of the above referenced ranges for the nominal length of the conductive wire(s) 140 are also possible (e.g., a nominal length of at least about 2.0 mm to no more than about 20 mm, or at least about 6 mm to more than about 14 mm).

In some embodiments, the conductive wire(s) 140 can be coupled to one or more lead wires to connect electrically the conductive wire(s) 140 with a connector similar to and/or the same as the connectors shown in FIGS. 7A, 7B, and 11. These connectors can electrically and operably coupled the conductive wire(s) 140 to a cardiac mapping system, a pulse field ablation (PFA) generator, or any other suitable device capable of generating 3D maps of the anatomy of a patient and/or delivering energy therapies. As described above, the lead wires can be made of a conductive metal such as copper, silver, nickel, aluminum, or alloys thereof. In some embodiments the lead wires can be made of the same material as the conductive wire(s) 140. Each lead wire can be routed through a shaft and/or interior volume of the conduit 160 from the distal end of the conduit 160 to a proximal end of the conduit 160, where the lead wire can be coupled to a connector. In some embodiments a lead wire coupled to a conductive wire 140 can have a nominal diameter of at least about 28 American Wire Gauge (AWG), at least about 29 AWG, at least about 30 AWG, at least about 32 AWG, at least about 34 AWG, at least about 36 AWG, at least about 38 AWG, at least about 40 AWG, or at least about 42 AWG, inclusive of all ranges and values therebetween. In some embodiments, a lead wire coupled to a conductive wire 140 can have a diameter of no more than about 42 AWG, no more than about 39 AWG, no more than about 37 AWG, no more than about 35 AWG, no more than about 33 AWG, no more than about 31 AWG, no more than about 29 AWG, or no more than about 28 AWG, inclusive of all ranges and values therebetween.

Combinations of the above referenced ranges for the nominal diameter of the lead wires of the ring electrodes are also possible (e.g., a nominal diameter of at least about 28 AWG to no more than about 42 AWG, or at least about 30 AWG to more than about 36 AWG.).

In some embodiments, the lead wires coupled to the conductive wires 140 can be insulated to prevent short circuiting and/or arching the conductive wire(s) 140 with the conductive element(s) 120 or with other conductive wire(s) 140. More specifically, the lead wires coupled to the conductive wire(s) 140 can be protected by a layer and/or coating of an electrical insulation material that covers the external surface of the lead wire from a contact point on the conductive wire 140 to a distal end of the conduit 160. The lead wires can then continue to be protected by a layer and/or coating of an electrical insulation material from the distal end of the conduit 160 to a proximal end of the conduit 160 where the lead wire is coupled to a connector. In some embodiments, the insulation on these lead wires can be and/or include heavy polyimide insulation having a nominal OD of about 0.0094 inches. In some embodiments, the insulation on these lead wires can be and/or include triple build polyimide insulation (e.g., three layers of polyimide insulation, with each layer having a nominal OD of about 0.0094 inches). Optionally, in some embodiments, these lead wires can be collectively routed within a lumen of the conduit 160 (e.g., an irrigation tube, channel and/or lumen) which is used to transport a saline solution or other liquid to the distal end of the conduit 160, as further described herein.

In some instances, the conductive wire(s) 140 can be configured to establish a potential and/or voltage across the conductive wire(s) 140 and a selected number of isolated conductive element(s) 120 on the contact assembly 110. Additionally or alternatively, in some instances a conductive wire 140 can be configured to establish a potential and/or voltage across another conductive wire 140 included in the contact assembly 110. The voltage applied via the conductive wire(s) 140 is configured to cause irreversible and/or reversible electroporation of cardiac cells. In some embodiments, the voltages applied by the conductive wire(s) 140 can be at least about 1,000 V, at least about 1,200 V, at least about 1,400 V, at least about 1,600 V, at least about 1,800 V, at least about 2,000 V, at least about 3,000 V, at least about 4,000 V, at least about 5,000 V, at least about 6,000 V, at least about 7,000 V, at least about 8,000 V, at least about 9,000 V, at least about 10,000 V, at least about 11,000 V, at least about 12,000 V, at least about 13,000 V, at least about 14,000 V, or at least about 15000 V, inclusive of all ranges and values therebetween. In some embodiments, the voltages applied by the conductive wire(s) 140 can be no more than about 15,000 Volts (V), no more than about 13,000 V, no more than about 11,000 V, no more than about 9,000 V, no more than about 7,000 V, no more than about 5,000 V, no more than about 3,000 V, no more than about 1,500 V, or no more than about 1,000 V, inclusive of all ranges and values therebetween.

Combinations of the above referenced ranges for the voltage established by the conductive wire(s) 140 are also possible (e.g., a voltage of at least about 3,000 V to no more than about 15,000 V, or at least about 9,500 V to more than about 13,000 V).

The conductive wire(s) 140 can be disposed on, extend from, and/or otherwise be coupled to a distal end-portion of the conduit 160. More specifically, the conductive wire(s) 140 can be disposed on a distal end-portion of the conduit 160 extending away from the distal end-portion of the conduit 160. Said in other words, the conductive wire(s) 140 can be mounted at a distal end of the conduit 160 and extend distally from the distal end of the conduit 160. In some embodiments, when the contact assembly 110 assumes the expanded configuration, the conductive wire(s) 140 can extend away from the distal end of the conduit 160 in multiple directions (e.g., the conductive wire(s) 140 extended distally from the distal end of the conduit 160 following non-parallel directions). In some embodiments, when the contact assembly 110 assumes the expanded configuration, the conductive wire(s) 140 extend away from the distal end of the conduit 160 such that each conductive wire 140 is oriented parallel to all the other conductive wire(s) 140. As described above, in some embodiments when the contact assembly 110 assumes the expanded configuration, each conductive wire 140 included in the contact assembly 110 is oriented parallel to each other and along a plane which can be brought into close proximity and/or direct contact with in vivo tissue of the heart of a patient (e.g., when the contact assembly 110 lays flat at close proximity or in direct contact with the tissue). In some embodiments, when the contact assembly 110 assumes the expanded configuration, each conductive wire 140 included in the contact assembly 110 can be oriented parallel to each other, and also with respect to a longitudinal axis defined by the conduit 160 from a distal end to a proximal end of the conduit 160, as further described herein. Alternatively, in some embodiments when the contact assembly 110 assumes the expanded configuration, each conductive wire 140 included in the contact assembly 110 can be oriented perpendicular to a longitudinal axis defined by the conduit 160. In some embodiments, when the contact assembly 110 assumes the expanded configuration, each conductive wire 140 included in the contact assembly 110 can be oriented perpendicular to the longitudinal axis defined by the conduit 160 and to a longitudinal axis defined by each supporting tube 130 included in the contact assembly 110.

In some embodiments, when the contact assembly 110 assumes the compressed configuration, the conductive wire(s) 140 can become collapsed and/or constrained along a longitudinal axis defined by the distal end of the conduit 160 at a very close proximity from each other such that the contact assembly 110 and all of their components have a form factor that fits within a delivery catheter or a sheath. For example, in some implementations, when the contact assembly 110 assumes the compressed configuration, the supporting tube(s) 130 (and other components of the contact assembly 110) are compressed against each other, and become aligned with the longitudinal axis defined by the conduit 160 at close proximity from the center of the conduit 160 such that a contour of the compressed contact assembly 110 has an outer diameter substantially similar to and/or the same as an outer diameter of the conduit 160, a suitable delivery catheter, or a sheath. In some embodiments, the conductive wire(s) 140 can be coupled to a connecting component 150 which in turn can include one or more wires that can mechanically couple the conductive wire(s) 140 with a dial disposed at a handle of the apparatus 100. The dial enables a user to adjust the tension on the one or more wires to flex and/or deform the conductive wire(s) 140, along with other components of the contact assembly 110, to better conform to the intracardiac tissue of the subject (e.g., to deform the contact assembly 110 to lay it flat on the intracardiac tissue).

The connecting component 150 can be an optional structure included in the contact assembly 110 to mechanically connect the supporting tubes(s) 130 and the conductive wire(s) 140 to facilitate placing the contact assembly 110 in the expanded configuration or in the compressed configuration in a reproducible manner. Said in other words, the connecting component 150 can include one or more segments mechanically coupled to the distal and/or the proximal ends of the supporting tube(s) 130 and the conductive wire(s) 140 to facilitate orienting the supporting tube(s) 130 and the conductive wire(s) 140 along a plane when the contact assembly 110 assumes the expanded configuration (e.g., enabling components of the contact assembly 110 to lay flat (or other preferred orientation) every time the contact assembly 110 assumes the expanded positions). Similarly, the connecting component 150 can include one or more segments mechanically coupled to the distal and/or the proximal ends of the supporting tube(s) 130 and the conductive wire(s) 140 to facilitate orienting the supporting tube(s) 130 and the conductive wire(s) 140 parallel to each other and at a very close proximity from each other (e.g., assuming a compressed configuration) such that the contact assembly 110 (and all of their components) have a form factor that fits within a delivery catheter or sheath. In some embodiments the connecting component 150 can be a single-piece, monolithic component, framework and/or or skeleton that provides structural support to the components of the contact assembly 110 (e.g., the conductive element(s) 120, the supporting tube(s) 130, and the conductive wire(s) 140), facilitating placing the contact assembly 110 in the expanded configuration or in the compressed configuration in a reproducible manner, as further described herein.

In some embodiments, in addition to facilitating placing the contact assembly 110 in the expanded configuration or in the compressed configuration in a reproducible manner, the connecting component 150 can be a structure configured to provide mechanical support to one or more components of the contact assembly 110, including one or more conductive element(s) 120, supporting tube(s) 130, and/or conductive wire(s) 140. In such embodiments, the connecting component 150 can be used to (1) mount and/or couple components of the contact assembly 110 and (2) facilitate placing the contact assembly 110 in the expanded configuration or in the compressed configuration in a reproducible manner. In particular, the connecting component 150 can provide mechanical support to the contact assembly 110 (and all of the components included therein) and at the same time facilitate orienting the supporting tube(s) 130 and the conductive wire(s) 140 (when present) along a plane when the contact assembly 110 assumes the expanded configuration, such that the conductive element(s) 120 disposed on the supporting tube(s) 130 and the conductive wire(s) lay flat and/or substantially flush (e.g., to maximize contact) against the tissue of a patient. Said in other words, the connecting component 150 can impart a planar orientation to the supporting tubes 130 and the conductive wires 140 disposed and/or mounted on the connecting component 150. In some implementations, for example, the connecting component 150 is a continuous and/or monolithic framework configured to impart a predefined orientation, in its expanded configuration, e.g., a substantially planar or planar orientation to the support tubes 130 and/or any other component disposed on or coupled to the connecting component 150. In some such implementations, the framework can effectively drive the predefined orientation of the contact assembly 110 as the contact assembly 110 is exposed, e.g., from a surrounding sheath, without, e.g., the need for any additional components, e.g., a coupler or fixation means (similar to existing devices that use independent or separate spines and/or loops that then need to be joined or coupled together to arrive at a desired orientation or shape). The connecting component 150 can also be configured to fold, flex, and/or contract to facilitate transitioning the contact assembly 110 into the compressed configuration, in which the supporting tube(s) 130 and the conductive wire(s) 140 become aligned parallel to each other and at a very close proximity from each other such that the contact assembly 110 (and all of its components) has a form factor that fits within a delivery catheter or sheath, facilitating advancing and/or withdrawing the contact assembly 110 through the vasculature of the patient (e.g., heart of the patient).

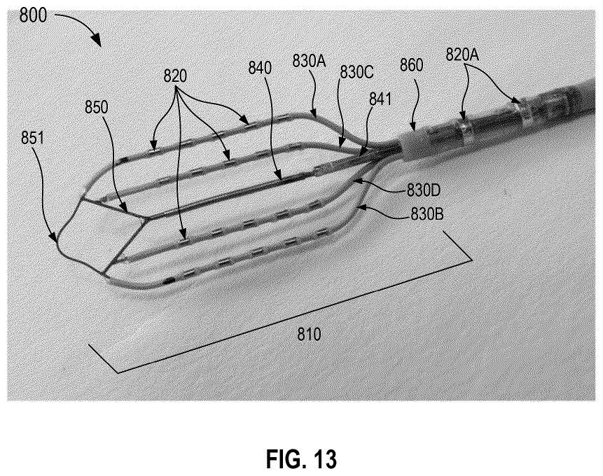

To facilitate folding, flexing, and/or contracting the connecting component 150, in some embodiments the connecting component 150 can include one or more features that can be collapsed when the contact assembly 110 is transitioned into the compressed configuration resulting in the alignment of the supporting tube(s) 130 and the conductive wire(s) 140 parallel to each other, e.g., at a very short distance. The one or more features can also be restored to their initial orientation (e.g., their orientation prior to being collapsed) when the contact assembly 110 is transitioned to the expanded configuration. For example, in some embodiments, the one or more features of the connecting component 150 can be and/or include a lateral support section configured to be disposed distal to or and/or support the distal end or termination of the supporting tubes 130. In some embodiments the lateral support section can include one or more lateral segments and/or angled segments (e.g., angled relative to the longitudinal axis of the conduit 160). The one or more lateral or angled segments can be disposed distal to or and/or supporting the distal end or termination of the supporting tubes 130 (see e.g., FIG. 13), and proximal to a distal-most terminal end of the contact assembly 110. The one or more lateral segments and/or angled segments can be folded to facilitate transitioning the contacting assembly 110 in the compressed configuration. Similarly, the one or more lateral segments and/or angled segments can be unfolded or returned to their orientation prior to being folded, to facilitate transitioning the contacting assembly 110 in the expanded configuration. In some embodiments the lateral support section of the connecting component 150 can include a V-shape (or similarly suitable shape) structure and/or feature that can be folded into itself (e.g., into a lineated configuration, e.g., with two arms of the V-shape moved into a parallel arrangement) to facilitate aligning the supporting tube(s) 130 and the conductive wire(s) 140 in close proximity such that they can fit within a delivery catheter or sheath, as further disclosed herein. Similarly, the default or at-rest V-shape feature can facilitate transition of the connecting component 150 (and any components included or coupled thereto) from it compressed configuration to its expanded configuration (e.g., from a lineated shape to the V-shape), as described in more detail herein. In some embodiments, the connecting component 150 can include a first portion and at least a second portion, with the second portion being structurally weaker than the first portion such that the connecting component 150 can be flexed concavely or convexly to transition the contact assembly 110 between the expanded configuration and the compressed configuration.

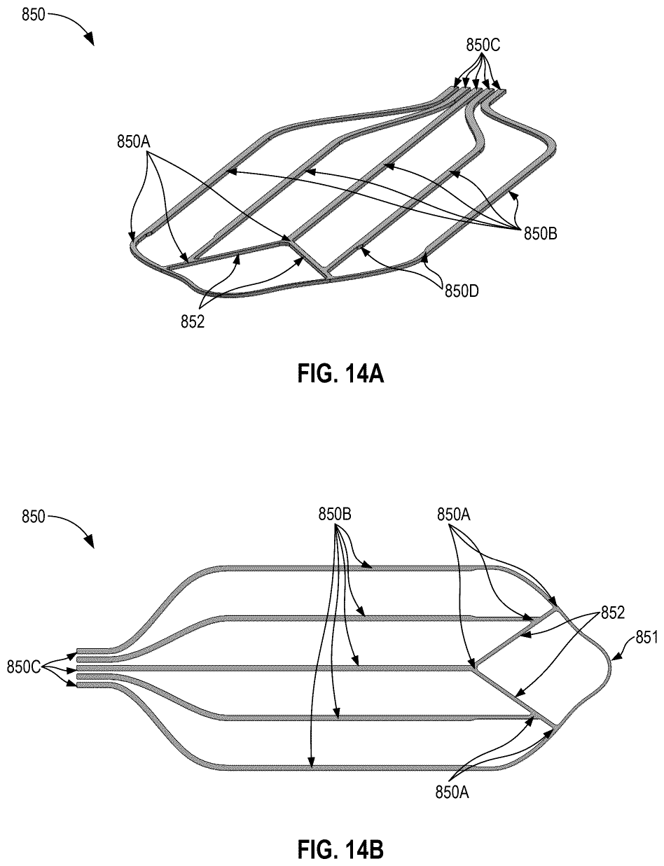

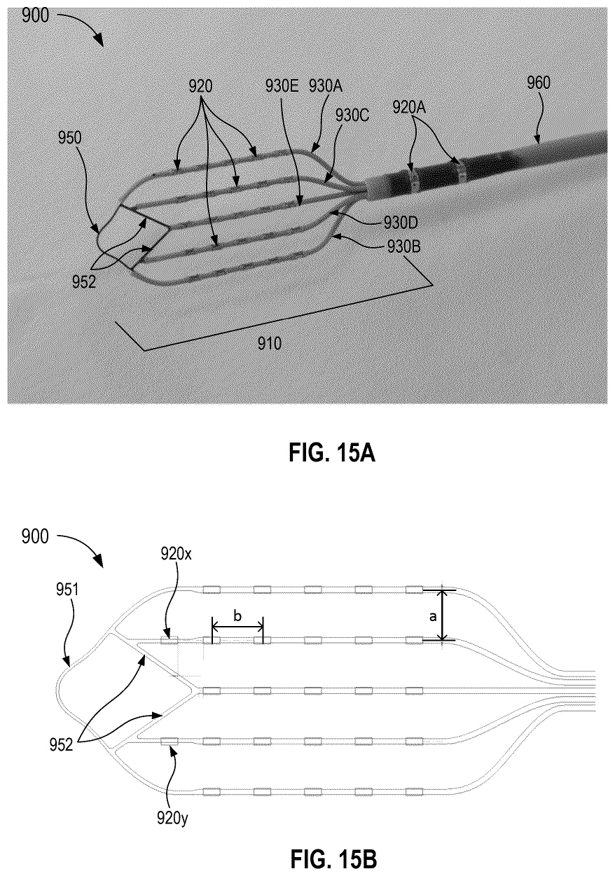

In some embodiments the connecting component 150 can be and/or include a skeleton-like single-piece or monolithic structure including a plurality of segments, sections, spines, tines, arms, or the like. The plurality of segments, sections, spines, tines, arms, or the like can also be referred to herein as a tube support section of the connecting component 150. Each arm from the plurality of arms can be sized and configured to support the weight of a supporting tube 130, with the supporting tube 130 having one or more conductive element(s) 120 coupled, mounted, and/or disposed on a surface of the supporting tube 130. In this manner, the connecting component 150 can serve as an understructure about which the supporting tubes 130 can be supported, and can be configured to extend from within the conduit 160 to a distal-most point or portion of the contact assembly 110 (e.g., beyond a distal-most point or portion of the supporting tubes 130). For example, in some embodiments each arm can have a cross-sectional area sized to be disposed within an interior volume of the supporting tubes 130. In use, each supporting tube 130 (with the one or more conductive element(s) 120 coupled thereto, e.g., disposed on its surface) can be arranged and/or slid over an arm from the connecting component 150 such that the weight of the supporting tube 130 and the conductive elements 120 is held by an arm of the connecting component 150. The plurality of arms (or a portion thereof) can be oriented parallel (or substantially parallel) to each other and along a plane (e.g., an in-plane and/or a co-planar orientation) when the contact assembly 110 assumes the expanded configuration, such that the conductive wire(s) 140 and the supporting tube(s) 130 with the conductive element(s) 120 disposed thereon, can lay flat and/or flush against the tissue of a patient. As disclosed above, in some embodiments the connecting component 150 can also include one or more features such as a lateral support section that allows folding and/or contracting the connecting component 150 such that the plurality of arms are oriented parallel to each other and at a very close proximity from each other (e.g., assuming a non-coplanar orientation), when the contact assembly 110 assumes the compressed configuration. Each arm from the plurality of arms can have a first end-portion, a mid-section, and a second end-portion opposite the first end-portion. In some embodiments, the first end-portion can stem from a tip and/or head of the connecting component 150. In some embodiments, the first end-portion can stem from the lateral support section of the connecting component 150, as shown for example, in FIGS. 14 and 15. The mid-section can be configured to assume the planar (or substantially planar) orientation when the contact assembly 110 assumes the expanded configuration, and the non-coplanar orientation when the contact assembly 110 assumes the compressed configuration. The second end-portion can be configured to converge at a region opposite to the tip and/or head of the connecting component 150 to facilitate coupling the plurality of arms to the conduit 160. More specifically, the second-end-portion of each arm from the plurality of arms can be disposed within the conduit 160 in close proximity with all other arms from the plurality of arms. The arms can exit the conduit 160 and then progressively become aligned according to the planar orientation (when the contact assembly 110 assumes the expanded configuration) or according to the non-coplanar orientation (when the contact assembly 110 assumes the compressed configuration), as further described herein with reference to the connecting component 850 and/or 950 shown in FIGS. 14-16.

In some embodiments, the connecting component 150 can include a plurality of segments, spines, sections, tines, arms, or the like, with each arm having a rectangular cross-sectional area. Alternatively, in some embodiments, the connecting component 150 can include a plurality of segments, spines, sections, tines, arms, or the like, with each arm having a triangular, circular, elliptical, and or any suitable polygonal cross-sectional area. In some embodiments, the connecting component 150 can include a tip and/or head, one or more features disposed adjacent to the tip and/or head (and configured to fold/unfold the contracting the connecting component 150), and any suitable number of segments, sections, arms, or the like. For example, in some embodiments the connecting component 150 can include at least about 2 arms, at least about 3 arms, at least about 4 arms, at least about 5 arms, at least about 6 arms, at least about 7 arms, at least about 8 arms, or at least about 10 arms, with each arm having a first end-portion that stems from the tip and/or head of the connecting component 150, or from the one or more features adjacent (e.g., proximal to) to the tip and/or head, and a second end-portion that can converge at a region opposite to the distal terminal end and/or tip of the connecting component 150 such that the arms can be coupled and/or inserted to the conduit 160. More specifically, in some embodiments the second end-portion of each arm of the connecting component 150 can be configured to be aligned parallel to the second end-portions of all other arms of the connecting component 150 and at a very close distance when they are not coupled to the conduit 160, as shown with the second end-portions 850C shown in FIGS. 14A and 14B. In use, the second end-portions of the arms can be disposed and/or placed inside an interior volume of the conduit 160 and secured therein (e.g., via an interference fit within the conduit 160. In that way, the second end-portions of the arms of the connecting component 150 can assume a non-planar orientation in which the second end-portions are oriented around a perimeter of the interior volume of the conduit 160 (e.g., a circumferentially-arranged orientation). In some implementations, such an arrangement may help minimize the diameter of the delivery sheath (not shown) and/or the conduit 160.

In some embodiments, the connecting component 150 can be and/or include a skeleton-like monolithic structure including a plurality of segments, sections, spines, tines, arms, or the like that provide mechanical support to one or more conductive wires 140. In such embodiments, the conductive wire(s) 140 can be hollow wire(s) defining an interior volume sized to accommodate an arm of the connecting component 150. In use, each conductive wire 140 can be slid over an arm from the connecting component 150 such that the weight of the conductive wire 140 is held (e.g., supported) by the arm of the connecting component 150. In some embodiments, the interior volume of each conductive wire 140 can be coated or covered with an insulating material to prevent the conductive wire 140 from being or becoming electrically coupled and/or connected to other components of the contact assembly 110 such as the conductive element(s) 120. Alternatively or additionally, in some embodiments an external surface of each arm of the connecting component 150 can be coated or covered with an insulating material to prevent the conductive wire 140 from being or becoming electrically coupled and/or connected to other components of the contact assembly 110 such as the conductive element(s) 120. In some embodiments, an external surface of the connecting component 150 can be coated or covered with one or more materials to impart and/or to improve a high dielectric constant to the connecting component 150 and/or the contact assembly 110. In some embodiments, the entire external surface of the connecting component 150 can be covered with the one or more materials that impart the high dielectric constant to the connecting component 150 and/or the contact assembly 110. Alternatively, in some embodiments, a portion and/or a fraction of the entire external surface area of the connecting component 150 can be covered with the one or more materials that impart the high dielectric constant to the connecting component 150 and/or the contact assembly 110. For example, in some embodiments a tip and/or head of the connecting component 150 and/or a mid-section of a plurality of arms of the connecting component 150 can be covered with the one or more materials that impart the high dielectric constant to the connecting component 150 and/or the contact assembly 110. In some embodiments, an external surface of the connecting component 150 can be coated or covered with one or more materials to impart and/or to improve a viscosity of the contact assembly 110, facilitating advancement of the contact assembly 110 through the vasculature a patient (e.g., of the heart of the patient). Similarly, in some embodiments, a portion and/or a fraction of the entire external surface area of the connecting component 150 (e.g., a tip and/or head of the connecting component 150 and/or a mid-section of a plurality of arms of the connecting component 150) can be covered with the one or more materials that impart the high viscosity to the contact assembly 110.

In some embodiments, the connecting component 150 can include a first portion and at least a second portion, with the second portion being structurally weaker than the first portion such that the connecting component 150 can be flexed concavely or convexly to transition the contact assembly 110 between the expanded configuration and the compressed configuration. In some embodiments, the skeleton-like monolithic structure of the connecting component 150 can include a first portion and/or set of regions and a second portion and/or set of regions, with the first portion and/or set of regions having a cross-sectional area smaller and/or weaker than a cross-sectional area of the second portion and/or set of regions. The difference in strength and/or cross-sectional areas of the first and second portions/regions of the skeleton-like monolithic structure, facilitates flexing concavely or convexly, and/or deforming the connecting component 150 such that the contact assembly 110 can be transitioned from a first configuration (e.g., relatively smaller delivery, constrained or compressed configuration) to a second configuration (e.g., e.g., relatively larger deployed, unconstrained configuration), as further disclosed herein.

In some implementations, when the contact assembly 110 assumes the compressed configuration all the components of the contact assembly 110 become compressed against each other towards or at the center of the conduit 160 (and aligned long a longitudinal axis defined by the conduit 160) such that a contour of the compressed contact assembly 110 has an outer diameter substantially similar to and/or the same as an inner diameter of the conduit 160, a suitable delivery catheter, or a sheath. In that way, the apparatus 100 can be advanced through the vasculature of patient similar to and/or the same as a conventional diagnostic catheter. In some embodiments, the connecting components 150 can include one or more segments configured to form a more rounded distal end of the apparatus 100 (e.g., a tip and/or head as disclosed above) to facilitate navigation through blood vessels of the patient, as further described herein. In some implementations, the connecting component 150 can be made of a solid material having sufficient flexibility and/or malleability to facilitate positioning each supporting tube 130 according to a predetermined orientation as the contact assembly 110 is transitioned between the compressed configuration and the expanded configuration. The connecting component 150 can be made of any suitable material including metals, metal alloys, polymers, and the like. In some embodiments, the connecting component 150 (or a portion thereof) can be made of and/or include polymeric materials having flexibility and deformability. For example, in some embodiments, the connecting component 150 can be made of a polymeric material such as PEEK. In some embodiments, the connecting component 150 (or a portion thereof) can be made of an insulating material including for example, amine thermoplastics such as Nylon, acrylonitrile-Butadiene-styrene ABS), polyacrylate, or the like. In some embodiments, the connecting component 150 (or a portion thereof) can be made of a conductive material having flexibility and/or malleability such as shape-memory metal or metal alloy materials including, for example, Nickel-Titanium (e.g., Nitinol), Copper-Aluminium-Nickel, Copper-Zinc-Aluminium, Iron-Manganese-Silicon, or the like or the like. In some embodiments, the connecting component 150 can include a distal portion and a proximal portion. In some embodiments, the distal portion can be made of a conductive material while the proximal portion is made of an insulating material. Alternatively, in some embodiments the distal portion can be made of an insulating material while the proximal portion is made of a conductive material. In some embodiments, unlike the supporting tube(s) 130, the connecting component 150 does not have any conductive element(s) 120 directly coupled or integrated thereto, and/or does not provide an external surface that can be used to directly couple, mount, attach, and or integrate a plurality of conductive element(s) 120. Instead, as disclosed above, in some embodiments the connecting component 150 can be a skeleton-like monolithic structure that provides mechanical support to one or more components of the contact assembly 110, including the conductive element(s) 120, the supporting tube(s) 130, and/or the conductive wire(s) 140 In such embodiments, the supporting tube(s) 130, and/or the conductive wire(s) 140 can be mounted and/or attached over the skeleton-like monolithic structure of the connecting component 150, as further disclosed herein. In some embodiments, the connecting component 150 does not define an interion lumen, volume, passage, cavity, and/or shaft that can accommodate subcomponents of the contact assembly 110 such as lead wires. In some embodiments, the connecting component 150 can include a projecting pointed part, convex section, tip, head, and/or prong (not shown in FIG. 1) disposed at a distal end of the contact assembly 110. The tip of the connecting component 150 can be made of a flexible, deformable and/or malleable material including, but not limited to, Aluminium, Aluminium alloys, Nylon, polyether block amides (e.g., Pebax®, Rnew®, Rilsan®, Polyamide 11®) or the like. In some embodiments, the tip of the connecting component 150 can be made of a material different from the material used for fabricating the rest of the connecting component 150. For example, in some embodiments, the tip of the connecting component 150 can be made of nitinol, aluminum, or an aluminum alloy while the rest of the connecting component 150 can be made of a polymer material such as PEEK. As described above, the tip of the connecting component 150 can be softer and/or more malleable than all the other portions and/or sections of the connecting component 150. In some embodiments, the tip of the connecting component 150 can be coupled and/or attached to the other portions and/or sections of the connecting component 150 by gluing, bonding, soldering, welding, or the like.

The conduit 160 can be any suitable structure defining an interior volume, cavity, lumen, shaft, or the like, that can be used to accommodate one or more components of the apparatus 100 including, for example, lead wires that electrically connect the conductive element(s) 120 and the conductive wire(s) 140 with one or more external devices and/or power sources. The conduit 160 can be made of any suitable material including, for example, metals, metal alloys, polymeric materials, and/or ceramic materials. The conduit 160 can be an elongated structure having a proximal end and a distal end and defining a longitudinal axis from the proximal end to the distal end. The conduit 160 includes one or more interior volumes, cavities, lumens, and/or shafts, oriented along the longitudinal axis of the conduit 160 from the proximal end (or proximal end-portion) to the distal end (or distal end-portion) of the conduit 160. In some embodiments, the interior volume of the conduit 160 can accommodate lead wires electrically coupled to one or more copper wound coils disposed on the supporting tubes 130. As described above, the lead wires coupled to the one or more copper wound coils can be routed from a connector located in the proximal end of the conduit 160 to the distal end of the conduit 160. After that, the lead wires can be routed through one or more of the supporting tube(s) 130 from the distal end of the conduit 160 to distal portion of the one or more supporting tube(s) 130, where the copper wound coils may be located. The copper wound coils can be used for electromagnetic localization to provide position information of the apparatus 100 within the body of a patient. In such embodiments, the conduit 160 can also accommodate one or more electromagnetic sensors configured to be used in conjunction with the copper wound coils. In some embodiments, the interior volume of the conduit 160 can accommodate one or more fiber optic elements. In some embodiments, the interior volume of the conduit 160 can accommodate a spring. The spring can be used to provide flexibility to the contact assembly 110 as the apparatus 100 is navigated through tortuous vasculature of the patient. In some embodiments, the interior volume of the conduit 160 can accommodate one or more thermocouples that enable taking temperature measurements at the distal end-portion of the conduit 160 or elsewhere in the apparatus 100. In some embodiments, the interior volume of the conduit 160 can accommodate one or more ultrasound transducer(s). In some embodiments, the interior volume of the conduit 160 can accommodate at least one video camera and/or sensor.

In some embodiments, the conduit 160 can also accommodate a tube disposed in the interior volume defined by the conduit 160 from the proximal end to the distal end of the conduit 160. This tube, which can also be referred to as an irrigation tube, channel, and/or lumen, can be sized and configured to transport one or more fluids (e.g., irrigation solutions) such as a saline solution from the proximal end of the conduit 160 to the distal end of the conduit 160. The irrigation channel can dispense the irrigation solution at the distal end of the conduit 160. More specifically, the irrigation tube can transport the irrigation solution to an area in the vicinity of the distal end of the conduit 160, where the supporting tube(s) 130 and the conductive wires(s) 140 extend away from the conduit 160. The irrigation solution can be dispensed with the purpose of preventing blood pooling, accumulating and/or clotting at the distal end-portion of the conduit 160.

In use, the apparatus 100 can be introduced via an incision (e.g., for access to the femoral artery) into the body of a patient. As described above, the contact assembly 110 of the apparatus 100 can assume a compressed configuration in which all the components included in the contact assembly 110 are aligned along the longitudinal axis of the conduit 160 resulting in an outer diameter of no more than about 8.5 Fr. This outer diameter of the contact assembly 110 in the compressed configuration is comparable and/or similar to the outer diameter of many diagnostic catheters. The apparatus 100 can then be advanced through the vasculature of the patient with the purpose of mapping, diagnosing, and/or delivering a treatment to one or more locations and/or regions of interest in the anatomy of the patient. In particular, the apparatus 100 can be advanced trough the vasculature of the patient while being disposed within a delivery catheter or sheath to reach a specific treatment location and/or region, frequently within the heart of the patient. The contact assembly 110 of the apparatus 100 can be transitioned from the compressed configuration to an expanded configuration in which the components of the contact assembly 110 are oriented parallel to each other and along a plane that can be brought into close proximity and/or direct contact with in vivo intracardiac tissue of the patient. In some instances, the transition of the contact assembly 110 between the compressed configuration and the expanded configuration can occur when the apparatus 100 is advanced with respect to the delivery catheter or sheath. In some instances, the transition of the contact assembly 110 between the compressed configuration and the In some instances, the transition of the contact assembly 110 between the compressed configuration and the expanded configuration can be assisted, at least partially, by a dial disposed on a handle of the apparatus 100, which is mechanically coupled to wires that can exert forces on the components of the contact assembly 110 to facilitate the transition of the contact assembly 110 between the compressed and the expanded configurations.

The conductive element(s) 120 can be used to measure and/or capture signals of the tissue with high fidelity, which can then facilitate the generation of a 3D map of the heart of the patient or region thereof. The conductive element(s) 120 can contact tissue of the patient to facilitate high fidelity signal acquisition to precisely identify cellular ectopic foci. In some embodiments the conductive element(s) 120 can be used to measure electrical data such as voltage, current, impedance, and/or depolarization. The voltages measured by the conductive element(s) 120 can be used to assess and/or diagnose the health of the tissue. Low voltage measurements can be used to identify areas of scar tissue while higher voltage measurements can be used to identify areas of healthy tissue that is depolarizing with each heartbeat. The electrical data measured by the conductive element(s) 120 can also be associated with three dimensional locations in space facilitate the reconstruction of 3D physiologic maps of the heart of the patient. Additionally, depolarization measurements gathered by the conductive element(s) 120 can also enable the mapping of electrical propagation through the heart. In this way, the apparatus 100 can be used for mapping the anatomy of the patient to guide the delivery of a therapy without the use of harmful ionizing radiation techniques such as X-Rays. As described above, the conductive element(s) 120 can include lead wires that connect the surface of the conductive element(s) 120 which are at close proximity or in direct contact with intracardiac tissue of the patient, with an external device such as an electrocardiogram recording system (EKG) configured to measure physiological parameters of the heart of the patient, and/or with a system designed to collect the electrical data and develop a 3D map of the heart of the patient.

In some instances, the apparatus 100 can also be used to deliver a therapy to tissue which has been identified as having clinical issues such as a cardiac arrhythmia. This delivery of therapy can occur seamlessly without the need to retract the apparatus 100 in order to introduce a separated (e.g., conventional) ablation catheter. As described above, the apparatus 100 can be used to establish a potential and/or voltage between the conductive wire(s) 140 and one or more conductive elements (120). These voltages enable delivering energy to cause irreversible and/or reversible electroporation of cardiac cells included in tissue of the heart of a patient. In some instances, the apparatus 100 can also be used to take one or more measurements using the conductive element(s) 120 to provide confirmatory post therapy delivery maps to assess if a particular therapy was delivered in the intended manner, the intended region, and whether further therapy is needed. The apparatus 100 can then be retracted trough the vasculature of the patient and removed once the procedure has been successfully conducted.

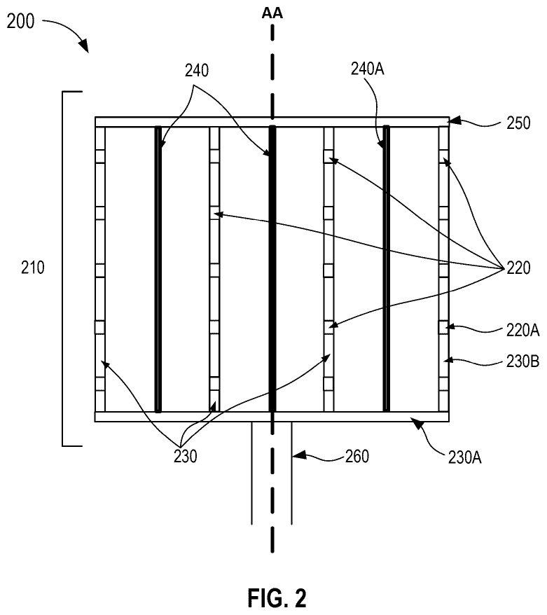

FIG. 2 shows a top view of an intracardiac apparatus 200 for mapping the anatomy of a patient and delivering ablation therapy (e.g., irreversible and/or reversible electroporation), according to an embodiment of the present disclosure. The intracardiac apparatus 200, which can also be referred to as the apparatus 200, or the catheter 200, can be the same or similar in form and/or function to the apparatus 100 described above with reference to FIG. 1. For example, as described above with reference to the apparatus 100, the apparatus 200 can be configured to be introduced via an incision into the body of a patient and be used to (1) measure electrical data that can be used to diagnose tissue of the patient and/or to generate a map (e.g., mapping) the anatomy of the patient, and (2) deliver a therapy in the form of irreversible and/or reversible electroporation ablation. The apparatus 200 includes a contact assembly 210 disposed on a distal end of a conduit 260. The contact assembly 210 includes a plurality of conductive element(s) 220, a plurality of supporting tube(s) 230, one or more conductive wire(s) 240, and a connecting component 250. In some implementations, portions and/or aspects of the apparatus 200 can be similar to and/or substantially the same as portions and/or aspects of the apparatus 100 described above with reference to FIG. 1. Accordingly, such similar portions and/or aspects may not be described in further detail herein.

FIG. 2 shows the contact assembly 210 disposed in a default, unconstrained, expanded configuration. As described above, in the expanded configuration, components of the contact assembly 210 are oriented parallel to each other and along a plane such that they can be brought into close proximity and/or direct contact with in vivo tissue of a specific treatment location and/or region on of a patient such as the heart of the patient. FIG. 2 shows the contact assembly 210 includes 20 conductive elements 220. However, in some implementations, the contact assembly 210 can include any suitable number of conductive elements 220. For example, in some implementations, the contact assembly 210 can include about 6, about 8, about 10, about 12, about 14, about 20, about 30, about 40, about 50, about 60, about 70, about 80, about 90, or about 100 conductive elements 220. The conductive elements 220 can be configured to contact tissue of the patient to facilitate high fidelity signal acquisition to precisely identify cellular ectopic foci. The conductive elements 220 can measure electrical data such as voltage, current, impedance, and/or depolarization. The voltages measured by the conductive elements 220 can be used to assess the health of the tissue of the heart of the patient. Low voltage measurements can be used to identify areas of scar tissue while higher voltage measurements can be used to identify areas of healthy tissue that is depolarizing with each heartbeat. The electrical data measured by the conductive elements 220 can be associated with three dimensional locations in space that facilitate the reconstruction of 3D physiologic maps of the heart of the patient. The conductive elements 220 can be ring electrodes. The ring electrodes can be mounted, attached, and/or integrated to an external surface of the supporting tubes 230. In some implementations, the ring electrodes can be characterized by a relatively small size including a nominal outer diameter (OD) of at least about 0.6 mm to no more than about 0.85 mm, and a nominal length of at least about 0.5 mm to no more than about 1.0 mm. Each conductive element 220 can be electrically coupled to a lead wire routed via the plurality of supporting tubes 230 to a distal end of the conduit 260, where the lead wires can be coupled to a connector disposed on a proximal end of the conduit 260, as further described herein.

In some implementations, the conductive elements 220 can be disposed on the supporting tubes 230 forming a two-dimensional flat array and/or matrix in which the conductive elements 220 are organized in columns and rows at specific and/or predetermined distances from each other when the contact assembly assumes the expanded configuration. More specifically, FIG. 2 shows each supporting tube 230 accommodates 5 conductive elements 220 placed along a longitudinal axis defined by the supporting tube 230 from a distal end of the supporting tube 230 to a proximal end of the supporting tube 230. The 5 conductive elements 220 disposed on or otherwise coupled to each supporting tube 230 can be placed at a fixed and/or constant spacing. Said in other words, two adjacent conductive elements 220 on a supporting tube 230 are separated by a fixed and/or predetermined distance along the longitudinal axis defined by the supporting tube 230. Conductive elements 220 disposed in adjacent supporting tubes 230 are also separated by a fixed and/or constant spacing, generating a 5×4 matrix. Alternatively, in some implementations the conductive elements 220 can be disposed on the supporting tubes 230 according to different distributions. For example, in some implementations the conductive elements 220 can be disposed on a first supporting tube 230 according to a first fixed and/or constant spacing from the distal end to the proximal end of the first supporting tube 230. A second supporting tube 230, located adjacent to the first supporting tube 230, can accommodate conductive elements 220 according to a second fixed and/or constant spacing different from the first spacing. In some implementations the second supporting tube 230 located adjacent to the first supporting tube 230 can accommodate a number of conductive elements 220 different form the number of conductive elements 220 disposed on the first supporting tube 230 (e.g., an alternating pattern of conductive elements 220). Alternatively, and/or optionally, in some implementations the conductive elements 220 can be disposed on random positions and/or locations along the supporting tubes 230.

FIG. 2 shows the contact assembly 210 includes a total of five supporting tubes 230. However, in some implementations the contact assembly 210 can include any suitable number of supporting tubes 230. For example, in some implementations the contact assembly 210 can include three supporting tubes 230 (e.g., 2 supporting tubes 230 oriented parallel to a longitudinal axis defined by the conduit 260, and one supporting tube 230 oriented perpendicular to the longitudinal axis defined by the conduit 260 and coupled to a distal end of the conduit 260). In the implementation shown in FIG. 2, the supporting tubes 230 include four supporting tubes 230 oriented parallel to a longitudinal axis defined by the conduit 260 (shown as axis AA in FIG. 2) and one supporting tube 230A coupled to the conduit 260 and oriented perpendicular to the longitudinal axis AA defined by the conduit 260. FIG. 2 shows the four parallel supporting tubes 230 extended distally from the supporting tube 230A, with the four parallel supporting tubes having the same fixed and/or constant length. The supporting tubes 230 (including the supporting tube 230A) provide an external surface that can be used to couple, mount, attach, and or integrate the conductive elements 220, and an interior lumen, volume, passage, cavity, and/or shaft that can accommodate various subcomponents of the contact assembly 210 such as lead wires connecting each conductive element 220 to a connector (not shown in FIG. 2) disposed on a proximal end of the conduit 260. More specifically, each one of the four parallel supporting tubes 230 shown in FIG. 2 can include a lumen that accommodates and routes lead wires electrically coupling each conductive element 220 with the connector disposed on the proximal end of the conduit 260. For example, as shown in FIG. 2, a conductive element 220A can have a lead wire having a first end-portion electrically coupled to the conductive element 220A. The lead wire can have a second portion adjacent to the first end-portion that is accommodated and/or routed through a lumen defined by the supporting tube 230B from the point on which the conductive element 220A is mounted to an intersection of the supporting tube 230B with the supporting tube 230A. The lead wire can have a third portion adjacent to the second portion of the lead wire, accommodated and/or routed through a lumen defined by the supporting tube 230 A from the intersection of the supporting tube 230B and the supporting tube 230A to a distal end of the conduit 260. The conduit 260 can also include a lumen that accommodates and/or routes the lead wire from the distal end of the conduit 260 to the proximal end of the conduit 260 where a connector is located.

In some implementations, the supporting tubes 230 can include copper wound coils (not shown in FIG. 2) that can be used with one or more electromagnetic position sensors disposed on the conduit 260 (also not shown in FIG. 2) to determine the position of the contact assembly 210 (or a portion thereof) with respect to the heart of a patient. In some implementations, one or more of the supporting tubes 230 can include a first portion and/or segment made of a strong material (e.g., a material having mechanical high strength, hardness, impact resistances and/or fracture toughness) and a at least one second portion and/or segment made of a material having weaker mechanical properties with respect to the material in the first portion. The second portions and/or sections having weaker mechanical properties can enable deforming the supporting tubes 230 to facilitate flexing the supporting tubes 230 concavely or convexly as needed. In some implementations, the supporting tubes 230 can include other subcomponents of the contact assembly 210 including, for example, fiber optic elements, energy storage devices such as springs, thermocouples, ultrasound transducers, video cameras and/or sensors, and the like. The supporting tubes 230 can be made of flexible material that are able to deform to accommodate small changes in the topography of the vasculature of the patient. In some implementations, the supporting tubes 230 can be made from flexible materials including polymers such as Polyether Ether Ketone (PEEK), Polypropylene (PP), Polystyrene (PS), Polycarbonate (PC), polyethylene terephthalate (PET) or the like. In some implementations, the supporting tubes 230 can be made of shape-memory metal or metal alloy materials including, for example, Nickel-Titanium (e.g., Nitinol), Copper-Aluminium-Nickel, Copper-Zinc-Aluminium, Iron-Manganese-Silicon, or the like.

The supporting tubes 230 can be configured to be compressed towards and/or against each other when the contact assembly 210 assumes the compressed configuration. As described above, in the compressed configuration the components included in the contact assembly 210 are collectively collapsed along the longitudinal axis defined by the conduit 260 (axis AA in FIG. 2) and at a short distance from each other, such that the apparatus 100 can be disposed within a delivery catheter or sheath. In some implementations, a cross-sectional area of the contact assembly 210 in the compressed configuration can be substantially similar to and/or the same as the outer diameter of the conduit 260. For example, in some implementations, a cross-sectional area of the contact assembly 210 in the compressed configuration can be smaller than or about the same as a delivery catheter no larger than about 8.5 Fr or less than about 8.5 Fr (e.g., 8 Fr, 7.5 Fr., etc.). The flexibility of the materials used to fabricate the supporting tubes 230 enables the natural compression of the supporting tubes 230 as the apparatus 200 navigates the vasculature of the patient. FIG. 2 shows the four parallel supporting tubes 230 are coupled to the connecting component 250 at a distal end of the four parallel supporting tubes 230. The connecting component 250 can facilitate orienting the four parallel supporting tubes 230 aligned along the longitudinal axis AA at a specific distance from each other when the contact assembly 210 is in the expanded configuration.

FIG. 2 shows the contact assembly 210 includes three conductive wires 240. However, in some implementations, the contact assembly 210 can include any suitable number of conductive wires 240. For example, in some implementations the contact assembly 210 can include only a single conductive wire 240. In other implementations, the contact assembly 210 can include 2, 4, 5 6 or more conductive wires 240. The conductive wires 240 can be made of electrically conductive materials such as copper, gold, platinum, or nitinol. Alternatively, in some implementations the conductive wires 240 can be made of a metal coated with platinum or including a radiopaque wound platinum wire. The conductive wires 240 facilitate establish a potential and/or voltage between a conductive wire 240 and one or more conductive elements 220. In some implementations, the conductive wires 240 can be configured to establish a potential and/or voltage with respect to other conductive wires 240. For example, a first conductive wire 240 can be configured to establish a potential and/or voltage with respect to a second conductive wire 240 disposed adjacent and/or next to the first conductive wire 240. In some implementations, the first conductive wire 240 can be configured to establish a potential and/or voltage with respect to a second conductive wire 240 that is not disposed adjacent to the first conductive wire 240. The potentials and/or voltages established by the conductive wires 240 enable delivering energy to cause irreversible and/or reversible electroporation of cardiac cells included in tissue of the heart of the patient. In some implementations, the conductive wires 240 are configured to deliver these voltages for a short period of time according to predetermined frequencies and/or voltage intensities. Said in other words, in some implementations the conductive wires 240 can be configured to deliver the voltages, and thus the irreversible electroporation ablation, according to any suitable pulse field ablation (PFA) methodology and/or treatment.

FIG. 2 shows the conductive wires 240 are aligned along the longitudinal axis AA defined by the conduit 260, with each conductive wire 240 being parallel to the other conductive wires 240. More specifically, the conductive wires 240 extend distally from the supporting tube 230A and in parallel with the other supporting tubes 230 included in the contact assembly 210. FIG. 2 shows the conductive wires 240 have the same nominal length. For example, in some implementations, the conductive wires 240 can be characterized by a nominal length of at least about 2.0 mm to no more than about 20 mm. In some implementations, the conductive wires 240 can have the same outer diameter (OD). In other implementations, each conductive wire 240 can have a different OD. In some implementations, the conductive wires 240 can be characterized by a nominal OD of at least about 0.2 mm to no more than about 1.0 mm.

The conductive wires 240 can be coupled to one or more lead wires (not shown) to connect electrically the conductive wires 240 with a connector similar to and/or the same as the connectors shown in FIGS. 7A, 7B, and 11. These connectors can electrically and operably coupled the conductive wires 240 to a cardiac mapping system, a pulse field ablation (PFA) generator, or any other suitable device capable of generating 3D maps of the anatomy of a patient and/or delivering energy therapies. The lead wires can be routed through the supporting tube 230A and through a shaft and/or interior volume of the conduit 260 from the distal end of the conduit 260 to a proximal end of the conduit 260, where the lead wire can be coupled to a connector (not shown). For example, the conductive wire 240A can be electrically coupled to a lead wire at the intersection of the conductive wire 240A and the supporting tube 230A. The lead wire can be routed from that intersection to a distal end of the conduit 260 using an internal lumen and/or volume defined by the supporting tube 230A. The lead wire can then be routed through an interior lumen and/or volume of the conduit 260 from the distal end of the conduit 260 to the proximal end of the conduit 260. In the case of the conductive wire 240 disposed in the central region of the contact assembly 210, that conductive wire 240 can be directly coupled to a lead wire at the distal end of the conduit 260. The lead wire can also be routed through an interior lumen and/or volume of the conduit 260 from the distal end of the conduit 260 to the proximal end of the conduit 260. The

The lead wires coupled to the conductive wires 240 can be insulated to prevent short circuiting and/or arching the conductive wires 240 with the conductive elements 220 or with other conductive wires 240. More specifically, the lead wires coupled to the conductive wires 240 can be protected by a layer and/or coating of an electrical insulation material that covers the external surface of the lead wire from a contact point on the conductive wire 240 to a distal end of the conduit 260. The lead wires can then continue to be protected by a layer and/or coating of an electrical insulation material from the distal end of the conduit 260 to a proximal end of the conduit 260 where the lead wire is coupled to a connector. In some embodiments, the insulation on these lead wires can be and/or include heavy polyimide insulation having a nominal OD of about 0.0094 inches.

FIG. 2 shows the connecting component 250 can be a structure similar in shape and/or form to the supporting tube 230A. In some implementations, unlike the supporting tube 230A (or the four parallel supporting tubes 230), the connecting component 250 does not have any conductive element(s) 220 directly coupled or integrated thereto, and/or does not provide an external surface that can be used to couple, mount, attach, and or integrate a plurality of conductive element(s) 220. In some embodiments, the connecting component 250 does not define an interion lumen, volume, passage, cavity, and/or shaft that can accommodate subcomponents of the contact assembly 210 such as lead wires for the conductive elements 220 or the conductive wire 240. The connecting component 250 can be disposed aligned perpendicular to the longitudinal axis AA defined by the conduit 260. The connecting component 250 can be coupled to a distal end of each one of the four parallel supporting tubes 230. FIG. 2 shows the connecting component 250 can also be coupled a distal end of the conductive wires 240. The connecting component 250 can be configured to facilitate placing the contact assembly 210 in the expanded configuration or in the compressed configuration in a reproducible manner. More specifically, the connecting component 250 includes a primary segments mechanically coupled to the distal end of the four parallel supporting tubes 230 and the three conductive wires 240 facilitating orienting the parallel supporting tubes 23 and the conductive wires 240 along a plane when the contact assembly 210 assumes the expanded configuration (e.g., enabling components of the contact assembly 210 to lay flat every time the contact assembly 210 assumes the expanded positions). In some implementations, the connecting component 250 can be a solid component made of a flexible and/or malleable material different from the materials used to fabricate the supporting tubes 230. In some implementations, the connecting component 250 can be made of material such as PEEK.

The conduit 260 can be used to accommodate one or more components of the apparatus 200 including, for example, lead wires that electrically connect the conductive elements 220 and the conductive wires 240 with one or more external devices and/or power sources via a connector. The conduit 260 is an elongated structure having a proximal end and a distal end, defining a longitudinal axis AA from the proximal end to the distal end. The conduit 260 includes an interior volume, cavity, lumen, and/or shaft, oriented along the longitudinal axis AA from the proximal end to the distal end of the conduit 260. In some implementations, the interior volume of the conduit 260 can accommodate lead wires electrically coupled to one or more copper wound coils disposed on the supporting tubes 230. As described above, the lead wires that are electrically coupled to the one or more copper wound coils can be routed from a connector located in the proximal end of the conduit 260 (not shown in FIG. 2) to the distal end of the conduit 260. After that, the lead wires of the copper wound coils can be routed through the supporting tube 230A and then through one or more of the four parallel supporting tubes 230 coupled to the supporting tube 230A. The lead wires of the copper wound coils can be routed through the one or more of the four parallel supporting tubes 230 from their intersection with the supporting tube 230A to a distal portion of the one or more supporting tube(s) 230, where the copper wound coils are located. The copper wound coils can be used for electromagnetic localization to provide position information of the apparatus 200 within the body of a patient. In such embodiments, the conduit 260 can also accommodate one or more electromagnetic sensors (not shown in FIG. 2) configured to be used in conjunction with the copper wound wires to determine the position of the apparatus 200 within the body of the patient.

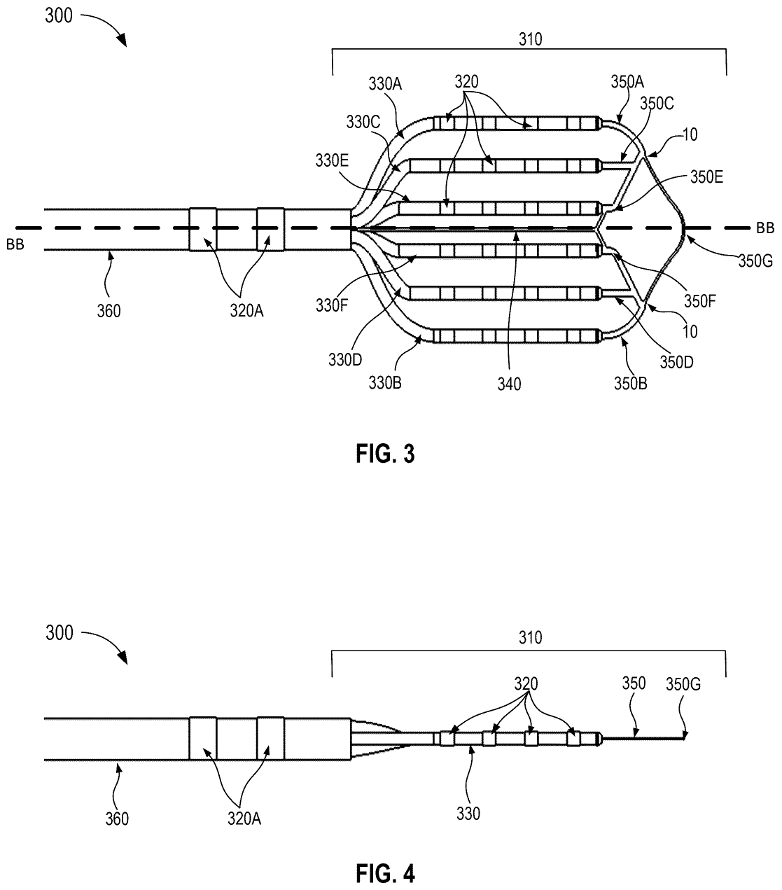

FIGS. 3, 4 and 5 show a top view, side view, and perspective view, respectively, of a an intracardiac apparatus 300 for mapping the anatomy of a patient and delivering ablation therapy (e.g., irreversible electroporation), according to an embodiment of the present disclosure. The intracardiac apparatus 300, which can also be referred to as the apparatus 300, or the catheter 300, can be the same or similar in form and/or function to the apparatus 100 and 200 described above with reference to FIGS. 1 and 2. For example, as described above with reference to the apparatus 100, the apparatus 300 can be configured to be introduced via an incision into the body of a patient and be used to (1) measure electrical data that can be used to diagnose tissue of the patient and/or to generate a map (e.g., mapping) the anatomy of the patient, and (2) deliver a therapy e.g., in the form of irreversible and/or reversible electroporation ablation. The apparatus 300 includes a contact assembly 310 disposed on a distal end of a conduit 360. The contact assembly 310 includes a plurality of conductive element(s) 320, a plurality of supporting tube(s) 330, one or more conductive wire(s) 340, and a connecting component 350. In some implementations, portions and/or aspects of the apparatus 300 can be similar to and/or substantially the same as portions and/or aspects of the apparatus 100 described above with reference to FIG. 1. Accordingly, such similar portions and/or aspects may not be described in further detail herein.

FIG. 3 shows the contact assembly 310 disposed in an expanded configuration. As described above, in the expanded configuration, components of the contact assembly 310 are oriented parallel to each other and along a plane such that they can be brought into close proximity and/or direct contact with in vivo tissue of a specific treatment location and/or region on of a patient such as the heart of the patient. FIG. 3 shows the contact assembly 310 includes 24 conductive elements 320. However, in some implementations, the contact assembly 310 can include any suitable number of conductive elements 320. For example, in some implementations, the contact assembly 310 can include about 6, about 8, about 10, about 12, about 14, about 20, about 30, about 40, about 50, about 60, about 70, about 80, about 90, or about 100 conductive elements 320.

The conductive elements 320 can be configured to contact tissue of the patient to facilitate high fidelity signal acquisition to precisely identify cellular ectopic foci. The conductive elements 320 can measure electrical data such as voltage, current, impedance, and/or depolarization. The voltages measured by the conductive elements 320 can be used to assess the health of the tissue of the heart of the patient. Low voltage measurements can be used to identify areas of scar tissue while higher voltage measurements can be used to identify areas of healthy tissue that is depolarizing with each heartbeat. The electrical data measured by the conductive elements 320 can be associated with three dimensional locations in space that facilitate the reconstruction of 3D physiologic maps of the heart of the patient. The conductive elements 320 can be ring electrodes. The ring electrodes can be mounted, attached, and/or integrated to an external surface of the supporting tubes 330 (e.g., supporting tubes 330A-F). In some implementations, the ring electrodes can be characterized by a relatively small sized including a nominal outer diameter (OD) of at least about 0.6 mm to no more than about 0.85 mm, and a nominal length of at least about 0.5 mm to no more than about 1.0 mm. Each conductive element 320 can be electrically coupled to a lead wire routed via the supporting tubes 330 to a distal end of the conduit 360, where the lead wires can be coupled to a connector disposed on a proximal end of the conduit 360, as further described herein.

The conductive elements 320 are disposed on the supporting tubes 330 forming a two-dimensional flat array and/or matrix in which the conductive elements 320 are organized in columns and rows at specific and/or predetermined distances from each other when the contact assembly assumes the expanded configuration. More specifically, FIGS. 3, 4 and 5 show each supporting tube 330 accommodates four conductive elements 320 placed along a longitudinal axis defined by the supporting tube 330 from a distal end of the supporting tube 330 to a proximal end of the supporting tube 330. In some implementations, the four conductive elements 320 disposed each supporting tube 330 can be placed at a fixed and/or constant spacing, similar to and/or the same as described above with reference to the conductive elements 220 and FIG. 2. Conductive elements 320 disposed in adjacent supporting tubes 330 are also separated by a fixed and/or constant spacing, thus generating a 4×6 matrix. For example, the four conducting elements 320 disposed on the supporting tube 330A are placed at a fixed and/or constant spacing from the distal end of the supporting tube 330A to the proximal end of the supporting tube 330A. The four conducting elements 320 disposed on the supporting tube 330B are also placed at the same fixed and/or constant spacing from the distal end of the supporting tube 330B to the proximal end of the supporting tube 330B. Consequently, the conducting elements 320 disposed on the supporting tubes 330A and 330B are also aligned along an axis perpendicular to a longitudinal axis defined by the conduit 360 (shown in FIG. 3 as axis BB) at a fixed and/or constant spacing, forming a matrix.

Alternatively, in some implementations the conductive elements 320 can be disposed on the supporting tubes 330 according to different distributions. For example, in some implementations the conductive elements 320 can be disposed on a first supporting tube 330 according to a first fixed and/or constant spacing from the distal end to the proximal end of the first supporting tube 330. A second supporting tube 330, located adjacent to the first supporting tube 330, can accommodate conductive elements 320 according to a second fixed and/or constant spacing different from the first spacing. In some implementations, the second supporting tube 330 located adjacent to the first supporting tube 330 can accommodate a number of conductive elements 320 different form the number of conductive elements 320 disposed on the first supporting tube 330 (e.g., an alternating pattern of conductive elements 320). Alternatively, and/or optionally, in some implementations the conductive elements 320 can be disposed on random positions and/or locations along the supporting tubes 330. FIGS. 3, 4 and 5 show the apparatus 300 can also include two conductive elements 320A disposed on an outer surface of the conduit 360. In some implementations, the conductive elements 320A can be used as reference and/or ground electrodes to facilitate taking measurements from the conductive elements 320 disposed on the supporting tubes 330 of the contact assembly 310.

FIG. 3 shows the contact assembly 310 includes a total of six supporting tubes 330 (e.g., 330A, 330B, 330C, 330D, 330E, and 330F). However, in some implementations the contact assembly 310 can include any suitable number of supporting tubes 330. For example, in some implementations the contact assembly 310 can include two supporting tubes 330 (each supporting tube 330 oriented parallel to the longitudinal axis BB defined by the conduit 360 and disposed between a conductive wire 340).

In the implementation shown in FIG. 3, the supporting tubes 330 include a first pair of outboard and/or exterior supporting tubes (e.g., supporting tubes 330A and 330B disposed on the external edges of the contact assembly 310), a second pair of inboard and/or interior supporting tubes 330 (e.g., supporting tubes 330E and 330F disposed adjacent to the conductive wire 340), and a third pair of supporting tubes 330 (e.g., middle supporting tubes 330C and 330D).

The supporting tubes 330 extend from a distal end of the conduit 360. More specifically, the supporting tubes 330 extended laterally and distally from the distal end of the conduit 360, with the pair of external supporting tubes 330A and 330B extending further away in the lateral direction with respect to the conduit 360, such that the external supporting tubes 330A and 330B define the terminal or outer-most edges of the contact assembly 310 when the contact assembly 310 assumes the extended configuration. The supporting tubes 330 provide an external surface that can be used to couple, mount, attach, and or integrate the conductive elements 320, and an interior lumen, volume, passage, cavity, and/or shaft that can accommodate various subcomponents of the contact assembly 310 such as lead wires connecting each conductive element 320 to a connector disposed on a proximal end of the conduit 360. The supporting tubes 330 can be coupled to one or more sections of the connecting component 350, as further described herein.

In some implementations, the supporting tubes 330 can include copper wound coils that can be used with one or more electromagnetic position sensors disposed on the conduit 360 to determine the position of the contact assembly 310 (or a portion thereof) with respect to the heart of a patient. In some implementations, one or more of the supporting tubes 330 can include a first portion and/or segment made of a strong material (e.g., a material having mechanical high strength, hardness, impact resistances and/or fracture toughness) and a at least one second portion and/or segment made of a material having weaker mechanical properties with respect to the material in the first portion. The second portions and/or sections having weaker mechanical properties can enable deforming the supporting tubes 330 to facilitate flexing the supporting tubes 330 concavely or convexly as needed. In some implementations, the supporting tubes 330 can include other subcomponents of the contact assembly 310 including, for example, fiber optic elements, energy storage devices such as springs, thermocouples, ultrasound transducers, video cameras and/or sensors, and the like. The supporting tubes 330 can be made of flexible material that are able to deform to accommodate small changes in the topography of the vasculature of the patient. In some implementations, the supporting tubes 330 can be made from flexible materials including polymers such as Polyether Ether Ketone (PEEK), Polypropylene (PP), Polystyrene (PS), Polycarbonate (PC), polyethylene terephthalate (PET) or the like. In some implementations, the supporting tubes 330 can be made of and/or include shape-memory metal or metal alloy materials including, for example, Nitinol, Copper-Aluminium-Nickel, Copper-Zinc-Aluminium, Iron-Manganese-Silicon, or the like or the like.

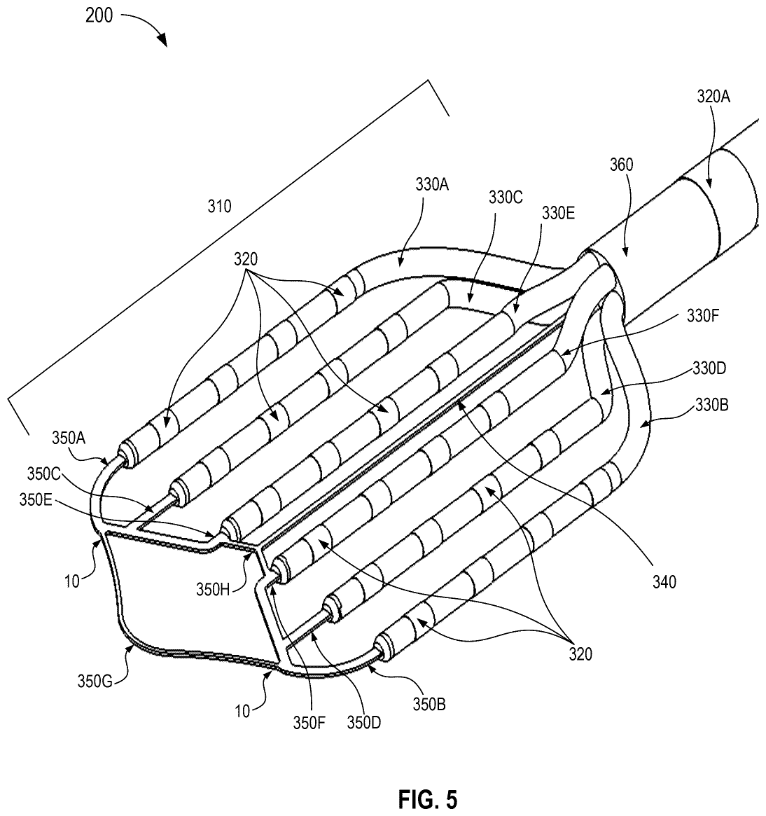

FIGS. 3 and 5 show the contact assembly 310 includes one conductive wire 340. However, in some implementations, the contact assembly 310 can include any suitable number of conductive wires 340. For example, in some implementations the contact assembly 310 can include 2, 3, 4, or more conductive wires 340.

The conductive wire 340 can be made of an electrically conductive material such as copper, gold, platinum, or nitinol. Alternatively, in some implementations the conductive wire 340 can be made of a metal coated with platinum or including a radiopaque wound platinum wire. The conductive wire 340 facilitates establishing a potential and/or voltage with one or more conductive elements 320. The potentials and/or voltages established by the conductive wire 340 enable delivering energy to cause irreversible and/or reversible electroporation of cardiac cells included in tissue of the heart of the patient. In some implementations, the conductive wire 340 is configured to deliver these voltages for a short period of time according to predetermined frequencies and/or voltage intensities. Said in other words, in some implementations the conductive wire 340 can be configured to deliver the voltages, and thus the irreversible electroporation ablation, according to any suitable pulse field ablation (PFA) methodology, technique, and/or treatment. In some instances, the delivery of irreversible and/or reversible electroporation therapy to a target tissue can generate lesions of a size proportional to the spacing between the electrodes that were used to establish the electroporation potential and/or voltage. In some implementations, the spacing between the conductive wire 340 and the conductive element(s) 320 used to generate an electroporation potential and/or voltage can be increased by selectively turning off conductive elements disposed adjacent and/or at a short distance from the conductive wire 340. In that way, an electroporation potential and/or voltage can be established over a larger region, generating a much larger lesion on the target tissue. For example, in some implementations the conductive elements 320 disposed on the supporting tube 330F and/or 330E can be used to establish an electroporation potential and/or voltage with the conductive wire 340. This potential can be used to deliver electroporation therapy over a target tissue generating a lesion. In order to increase the size of this lesion, in some implementations the conductive elements 320 disposed on the supporting tube 330F and/or 330E can be turned off and instead the electroporation potential can be established between the conductive wire 340 and the conductive elements 320 disposed on the supporting tube 330C and/or 330D. The larger spacing between the conductive wire 340 and the supporting tubes 330C and/or 330D can result in electroporation lesions having larger size compared to the lesion generated when using the conductive elements 320 disposed on the supporting tubes 330F and/or 330E. Similarly, turning off the conductive elements 320 disposed on the supporting tube 330F and/or 330E and instead generating the electroporation potential between the conductive wire 340 and the conductive elements 320 disposed on the supporting tubes 33A and/or 330B can result in even larger lesions. In this manner, an operator can selectively (in real-time during the procedure, and/or prior to the procedure) adjust (e.g., increase and/or decrease) the ablation area, shape, and/or footprint to tailor the therapy to a particular patient, tissue, disease-state, etc. In some implementations, the adjustment may be based on and/or in response to signal(s) received at and/or generated by the apparatus 300, and/or mapping data generated at least in part by data collected by the apparatus 300.

FIGS. 3 and 4 show the conductive wire 340 is aligned along the longitudinal axis BB defined by the conduit 360, extending distally from a distal end of the conduit 360. FIG. 3 shows the conductive wire 340 is oriented parallel to the supporting tubes 330A-F. Furthermore, FIG. 5 displays that the conductive wire 340 has a nominal length (measured from the distal end of the conduit 360) such that a distal end of the conductive wire 340 is disposed slightly proximal to the distal end of each one of the supporting tubes 330 (e.g., the distal end of the supporting tubes 330A-F is disposed distally to the distal end of the conductive wire 340). Said in other words, in some implementations the nominal length of the conductive wire 340 can be shorter than the length of the supporting tubes 330 along the longitudinal axis BB from the distal end of the conduit 360. In some implementations, the conductive wire 340 can be characterized by a nominal length of at least about 2.0 mm to no more than about 20 mm, and a nominal outer diameter (OD) of at least about 0.2 mm to no more than about 1.0 mm.

FIG. 5 shows the connecting component 350 includes multiple sections and/or portions that collectively couple the distal end of all the supporting tubes 330. More specifically, each supporting tube 330 is coupled at a distal end of the supporting tube 330 to a section and/or portion of the connecting component 350. For example, as shown in FIG. 5 the distal ends of the supporting tubes 330B, 330D, and 330F are coupled to the sections 350B, 350D, and 350F, respectively. Similarly, the distal ends of the supporting tubes 330A, 330C, and 330E are coupled to the sections 350A, 350C, and 350E, respectively. In some embodiments, unlike the supporting tubes 330, the connecting component 350 does not have any conductive element(s) 320 directly coupled or integrated thereto and/or does not provide an external surface that can be used to couple, mount, attach, and/or integrate a plurality of conductive elements 320. In some implementations, the connecting component 350 does not define an interior lumen, volume, passage, cavity and/or shaft that can accommodate subcomponents of the contact assembly 310 such as lead wires. As shown in FIG. 5, the sections 350A-F can be connected and or coupled together forming a concave shape that joins all the distal ends of the supporting tubes 330 with the distal end of the conductive wire 340 at a point and or location 350H, which is proximal to the distal end of the supporting tubes 330. The connecting component 350 can also include a projecting pointed part, convex section, tip, head, and/or prong 350G disposed at a distal end of the apparatus 300. This distal end can be configured to be atraumatic. FIG. 5 shows the tip 350G of the connecting component 350 is disposed distally to the point or location 350H where the supporting tubes 330 are joined together via the multiple sections and/or portions (e.g., 350A-F) of the connecting component 350. Furthermore, FIG. 3, FIG. 4 and FIG. 5 show that the tip 350G of the connecting component 350 can be attached to the distal end of the contact assembly 310 and more specifically to the sections and/or portions 350A-F of the connecting component 350. In some implementations, the tip 350G can be coupled and/or attached to the remaining sections and/or portions of the component 350 by welding, bonding (gluing), or over-molding with plastic at the joining points 10, as shown in FIGS. 3 and 5. In some implementations the tip 350G can be made of a material different from the material used to fabricate all the other sections and/or portions of the connecting component 350 (e.g., sections 350A-F). In some embodiments, the tip 350G can be made of a material having more flexibility and/or malleability compared to the material used to fabricate the other sections and/or portions of the connecting component 350. For example, in some implementations, the tip 350 G can be made of a material such as aluminum, aluminum alloy, Nitinol, Nylon, or commonly used medical plastic such as poly ether block amides (commonly known as PEBAX). In some implementations, the tip 350G of the connecting component 350 can be made of a conductive material such as nitinol, while all the other sections and/or portions of the connecting component 350 (e.g., 350A-F) are made of an insulating material such as nylon. Alternatively, in some implementations, the tip 350G of the connecting component 350 can be made of an insulating material such as nylon, while all the other sections and/or portions of the connecting component 350 (e.g., 350A-F) are made of a conductive material such as nitinol. In some implementations, the shape, softness and flexibility of the tip 350G facilitate atraumatically traversing blood vessels without perforation at the distal point of contact (e.g., first contact) with the tissue. In use, for example, the apparatus 300 can be advanced through the patient's vessel(s) within a delivery catheter (i.e., transcatheter delivery), and then the contact assembly 310 and/or a portion thereof, such as, e.g., the tip 350G, once in the heart, can be advanced and exposed from the delivery catheter (not shown), and the contact assembly 310 or portion thereof can then be navigated or advanced into desired blood vessels (e.g., pulmonary veins) extending from the heart chambers. The tip 350G as described in more detail here can be configured for such navigation within the heart and surrounding vessels without inadvertently damaging any of the surrounding anatomy.

The conduit 360 can be used to accommodate one or more components of the apparatus 300 including, for example, lead wires that electrically connect the conductive elements 320 and the conductive wires 340 with one or more external devices and/or power sources via a connector. The conduit 360 is an elongated structure having a proximal end and a distal end, defining a longitudinal axis BB (shown in FIG. 3) from the proximal end to the distal end. The conduit 360 includes an interior volume, cavity, lumen, and/or shaft, oriented along the longitudinal axis BB from the proximal end to the distal end of the conduit 360. In some implementations, the interior volume of the conduit 360 can accommodate lead wires electrically coupled to one or more copper wound coils disposed on the supporting tubes 330. As described above, the lead wires coupled to the one or more copper wound coils can be routed from a connector located in the proximal end of the conduit 360 (not shown in FIGS. 3-5) to the distal end of the conduit 360. After that, the lead wires can be routed through one or more of the supporting tubes 330 a distal portion of the one or more supporting tube(s) 330, where the copper wound coils may be located. The copper wound coils can be used for electromagnetic localization to provide position information of the apparatus 300 within the body of a patient. In such embodiments, the conduit 360 can also accommodate one or more electromagnetic sensors (not shown in FIGS. 3-5) configured to be used in conjunction with the copper wound coils to determine the position of the apparatus 300 within the body of the patient.

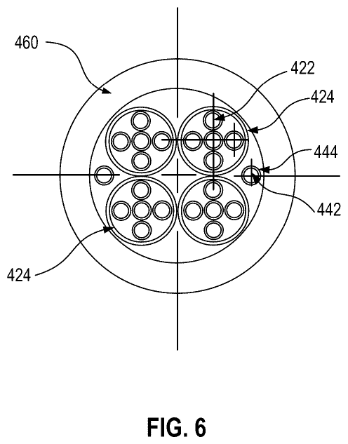

FIG. 6 shows a cross-sectional front view of a conduit 460 of an intracardiac apparatus displaying electrical connections for conductive elements and conductive wires, according to an embodiment of the present disclosure. FIG. 6 shows the conduit 460 defines an interior volume, cavity, lumen, and/or shaft that can be used to accommodate multiple components of an intracardiac apparatus. For example, as shown in FIG. 6, the conduit 460 can accommodate multiple lead wires 422 that are electrically coupled to conductive elements such as ring electrodes disposed on a contact assembly of the intracardiac apparatus. In some implementations, each lead wire 422 can be surrounded and/or coated with an insulating material (not shown in FIG. 6). Additionally, in some implementation, the lead wires that are coupled to the conductive elements can be collectively grouped on one or more tubes 424 made of heavily insulating materials such as heavy-duty polyimide, as shown in FIG. 6. The conduit 460 can also include lead wires 442 electrically coupled to one or more conductive wires of an intracardiac apparatus. As shown in FIG. 6, the lead wires 442 can be surrounded and/or coated with insulating materials 444 to prevent short circuiting and/or arching the conductive wires with one or more conductive elements included in the intracardiac apparatus.

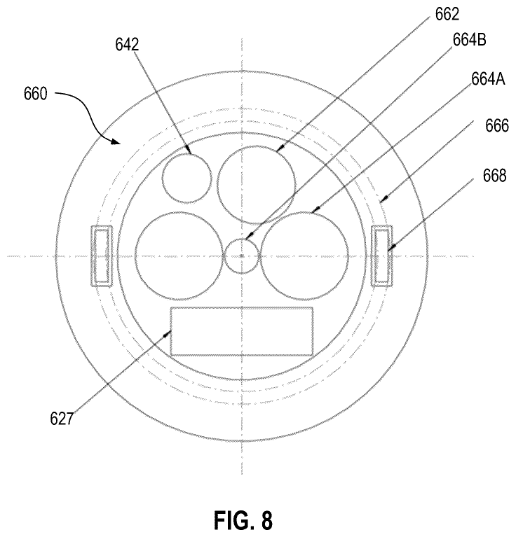

FIGS. 7A and 7B show example connectors 426 and 526 used to electrically connect a plurality of conductive elements of an intracardiac apparatus (similar to and/or the same as the apparatus 100, 200, or 300 described above with reference to FIGS. 1-5) with an external device such as a power source, an EKG device, a potentiostat, or the like. The connectors 426 can be grouped along a module 427 which can be disposed within a proximal end of a conduit, similar to the conduit 160, 260, 360 or 460 described above. In particular, the connectors 426 shown in FIG. 7A are a series of individual pin connectors which can be electrically coupled to the lead wires 422 shown in FIG. 6. The connectors 426 are integrated into the structure 427 according to a circular design (e.g., circular connector structure 427). The connector structure 427 can be disposed within a lumen defined by the conduit 460. The connector structure 427 can be oriented perpendicular to a longitudinal axis defined by the conduit 460, similar to the axis AA and axis BB shown in FIGS. 2 and 3 respectively. The connector structure 427 includes a region 424 made of insulating materials which prevent short circuiting the lead wires coupled to the conductive elements and the conductive wires of the intracardiac apparatus. The connectors 526 shown in FIG. 7B presents an alternative embodiment in which the connectors 526 are integrated into a structure 527 according to a rectangular shape and/or design. The connector structure 527 can be disposed within a lumen defined by conduit of an intracardiac apparatus. The connector structure 527 can be oriented perpendicular to a longitudinal axis defined by the conduit, as schematically illustrated in FIG. 8. The connector structure 527 also includes a region 524 made of insulating materials which prevent short circuiting the lead wires coupled to the conductive elements and the conductive wires of the intracardiac apparatus.

FIG. 8 shows a cross-sectional front view of a conduit 660 of an intracardiac apparatus displaying multiple channels for accommodating electrical connections for conducting elements and conductive wires; flowing irrigation solutions; and housing electromagnetic sensors and deflection elements, according to an embodiment of the present disclosure. FIG. 8 shows the conduit 660 includes an interior volume, cavity, lumen, shaft, or the like, that can be used to accommodate several components of an intracardiac apparatus substantially similar to and/or the same as the apparatus 100, 200, and 300 described above with reference to FIGS. 1-5. The conduit 660 includes an interior lumen and/or volume that can accommodate multiple components such as for example, lead wires that electrically connect conductive element(s) and the conductive wire(s) of the intracardiac apparatus with one or more external devices such as an EKG device, a cardiac mapping system, a pulse field ablation (PFA) generator, or any other suitable device capable of generating 3D maps of the anatomy of a patient and/or delivering energy therapie. In particular, FIG. 8 shows the conduit 660 includes a tube 622 that houses a plurality of lead wires 642 that are electrically coupled to one or more conductive wires of the intracardiac apparatus. As described above, the tube 622 can include one or more layers of insulation disposed around the external perimeter of the tube 622, sized and configured to prevent the shorting and/or arching of the conductive elements and conductive wires included in the intracardiac apparatus. The conduit 660 can also include a rectangular connector 627, similar to and/or the same as the rectangular connector 527 shown in FIG. 7. The connector 627 can receive a plurality of lead wires electrically coupled to multiple conductive elements included in the intracardiac apparatus. The conduit 660 can also accommodate an irrigation tube, channel, and/or lumen 662 sized and configured to transport one or more fluids (e.g., irrigation solutions) such as saline solutions from a proximal end of the conduit 660 to a distal end of the conduit 660. The irrigation channel 662 can dispense the irrigation solution at the distal end of the conduit 660. More specifically, the irrigation tube can transport the irrigation solution to an area in the vicinity of the distal end of the conduit 660, where the supporting tube(s) and the conductive wires(s) of the intracardiac apparatus extend away from the conduit 660. The irrigation solution can be dispensed with the purpose of preventing blood pooling, accumulating and/or clotting at the distal end-portion of the conduit 660.

The conduit 660 can also include and/or accommodate two electromagnetic sensors 664A which can be used together with a plurality of copper wound coils disposed on a contact assembly of the intracardiac apparatus. The electromagnetic sensors 664A are disposed symmetrically with respect to the center of the conduit 660, are used to determine relative positions of the intracardiac apparatus (or portions thereof) when copper wound coils disposed in the supporting tubes of the intracardiac apparatus are passed a current signal. FIG. 8 shows the conduit 660 can also accommodate lead wires electrically coupled to one or more copper wound coils disposed on the intracardiac apparatus. As described above, the lead wires coupled to the one or more copper wound coils can be routed from a connector located in the proximal end of the conduit 660 (not shown in FIG. 8) to a distal end of the conduit 660. After that, the lead wires can be routed through one or more of the supporting tubes of the intracardiac apparatus to a distal portion of the one or more supporting tube(s) of the intracardiac apparatus, where the copper wound coils may be located. The copper wound coils can be used for electromagnetic localization to provide position information of the apparatus 300 within the body of a patient. The conduit 660 can also include a pull ring 66 and a pull wire 668 which are mechanically coupled to the supporting tube(s) and/or supporting wires, or the connecting component of the intracardiac apparatus. The pull wires can be attached to and/or coupled to a dial disposed at a handle of the intracardiac apparatus. The dial enables a user to adjust the tension on the pull wires 668 to flex and/or deform the supporting tube(s), along with other components of a contact assembly of the intracardiac apparatus to better conform to the intracardiac tissue of the subject (e.g., to deform the contact assembly to lay flat on the intracardiac tissue).

FIG. 9 shows a handle 770 of an intracardiac apparatus (or coupled to the intracardiac apparatus) substantially similar to and/or the same as the intracardiac apparatus 100, 200, and 300 disposed above with reference to FIGS. 1-5. The handle 770 provides a means of connecting the intracardiac apparatus to external systems via a cable output connector 780, which typically located at the most proximal end of the handle 770, as shown in FIG. 9. FIG. 9 shows the cable output connector 780 can be coupled to the handle 770 by inserting the cable output connector 780 along the direction CC shown in FIG. 9. Conventional systems include a cable output connector designed with a connection plane that is oriented perpendicular to a longitudinal axis defined by the handle, a shown for example, with the connector structures 427 and 527 shown in FIGS. 7A and 7B respectively. This connection plane is typically 10-20 mm in diameter and thus limits the number and distance between connection pins/socket contacts of the connector. As described above, the need to provide robust insulation for the conductive element(s) and the conductive wire(s) of an intracardiac apparatus imposes a significant challenge to the integration of a catheter that integrates irreversible and/or reversible electroporation ablation capabilities with mapping capabilities. The handle 770 can also include a dial 772 that enables a user to adjust the tension on the one or more wires to flex and/or deform the supporting tube(s) and/or other components of the intracardiac apparatus, to better conform to the intracardiac tissue of the subject (e.g., to deform the contact assembly of the intracardiac apparatus to lay flat on the intracardiac tissue).

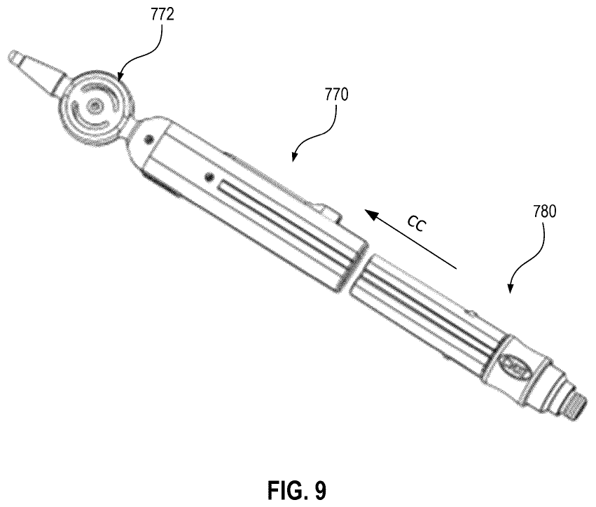

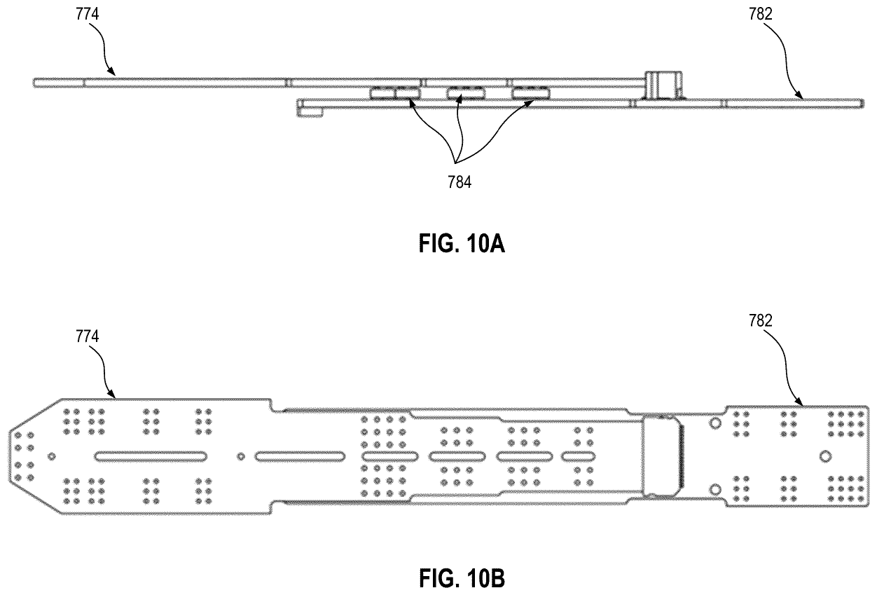

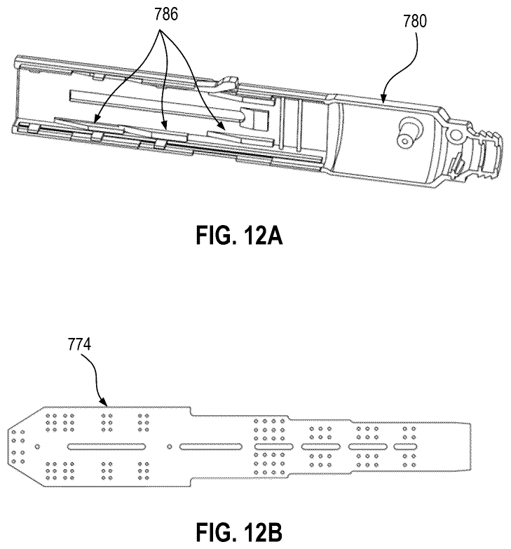

FIGS. 10A and 10 B show that the handle 770 can include a connector 774 that rotates the connection plan to be oriented parallel to the longitudinal axis defined by the handle. FIG. 11 shows the handle 770 can include a connector 774 in the form of a printed circuit board (PCB). The connector 774 provides a connection plane that is proportional to the width of the handle 770 (e.g., 10-25 mm) and the length of insertion depth (e.g., 20-130 mm), enabling sufficient separation of the contacts 784 for the conductive element(s) and conductive wire(s) of the intracardiac apparatus. FIG. 12 shows the cable output connector 780 can include a housing 786 that includes staggered ramps 786 that direct the catheter printed circuit board 774 downward during insertion and provides increased mating contact force through the area of contacts.

FIG. 13 shows a perspective view of an intracardiac apparatus 800 for mapping the anatomy of a patient and delivering ablation therapy (e.g., irreversible and/or reversible electroporation), according to an embodiment of the present disclosure. The intracardiac apparatus 800, which can also be referred to as the apparatus 800, or the catheter 800, can be the same or similar in form and/or function to the apparatus 100, 200, and 300 described above with reference to FIGS. 1-5. For example, as described above with reference to the apparatus 100, the apparatus 800 can be configured to be introduced via an incision into the body of a patient and be used to (1) measure electrical data that can be used to diagnose tissue of the patient and/or to generate a map (e.g., mapping) the anatomy of the patient, and (2) deliver a therapy in the form of irreversible and/or reversible electroporation ablation. The apparatus 800 includes a contact assembly 810 disposed on or extending from a distal end of a conduit 860. The contact assembly 810 includes a plurality of conductive element(s) 820, a plurality of supporting tube(s) 830, a conductive wire 840, and a connecting component 850. In some implementations, portions and/or aspects of the apparatus 800 can be similar to and/or substantially the same as portions and/or aspects of the apparatus 100, 200, and/or 300 described above with reference to FIGS. 1-5. Accordingly, such similar portions and/or aspects may not be described in further detail herein.

FIGS. 13, 14A and 14B show the connecting component 850 is a skeleton-like monolithic structure including a plurality of segments, sections, spines, tines, arms, or the like that provide mechanical support to the conductive elements 820, the supporting tubes 830, and the conductive wire 840. More specifically, the connecting component 850 includes a tip and/or head 851, a lateral or angled support section (also referred to herein as a V-shape feature 852), and five different segments, sections, spines, tines, arms, or the like, collectively referred to herein as a tube support section, that stem from the V-shape feature 852 of the connecting component 850, as further described herein. The five arms of the connecting component 850 are sized and configured to provide mechanical support to the conductive wire 840 and the supporting tubes 830A, 830B, 830C and 830D (e.g., the supporting tubes 830 A-D). The conductive wire 840 and the supporting tubes 830 A-D can be coupled and/or mounted on the arms of the connecting component 850 by introducing each arm within an interior volume and/or lumen defined by the conductive wire 840 and the supporting tubes 830 A-D. Said in other words, the conductive wire 840 can be moved along or slid over one of the arms of the connecting component 850 (e.g., a central arm of the connecting component 850 shown in FIG. 13. Similarly, each one of the supporting tubes 830 A-D can be moved along or slid over the arms of the connecting component 850. FIG. 13 shows each supporting tube 830 includes conductive elements 830 disposed on a surface of the supporting tube 830. As disclosed above, in some embodiments each conductive element 820 can include a lead wire coupled to and/or attached to the conductive element 820 to connect electrically the conductive element 820 with a connector similar to and/or the same as the connectors shown in FIGS. 7A, 7B, and 11. The lead wires of the conductive elements 820 shown in FIG. 13 can be accommodated within an interior volume of the supporting tubes 830 that is not occupied by the arms of the connecting component 850. Said in another way, each lead wire can be placed within an annular region and/or volume defined as the interior volume of a supporting tube 830 that is not occupied by an arm of the connecting component 850. In some embodiments, each lead wire can be coated or covered with an insulating material to prevent short-circuiting one or more conductive elements 820.

FIGS. 14A and 14B show a detailed perspective and a top view of the connecting component 850, illustrating that each arm of the connecting component 850 has a first end-portion 850A, a mid-section 850B, and a second end-portion 850C opposite to the first end-portion 850A. Additionally, FIGS. 14A and 14B show the connecting component 850 also includes a V-shape feature 852 disposed proximal to and connected to a tip 851 and/or head of the connecting component 850. The V-shape feature 852 can be configured to fold and/or contract to facilitate transitioning the contact assembly 810 between an expanded configuration and a compressed configuration, as further described herein. FIGS. 14A and 14B show the first end-portion 850A of each arm stems from the V-shape feature 852. The mid-section 850B of each arm can be configured to be oriented parallel to the mid-section 850B of the other arms and along a plane (e.g., assuming an in-plane and/or planar orientation) when the contact assembly 810 assumes an expanded configuration, such that the conductive wire 840 and the supporting tubes 830 A-D with the conductive element(s) 820 disposed thereon, can lay flat and/or flush against tissue of a patient, facilitating delivering a treatment to a target region in the body of a patient and/or generating a 3-D map of the patient's anatomy. The mid-section 850B of each arm can also be configured to be aligned parallel to each other and at a very close proximity from other mid-sections 850B of other arms (e.g., assuming a non-coplanar orientation) when the contact assembly 810 assumes an compressed configuration, such that the contact assembly 810 (and all of their components) has a form factor that fits within a delivery catheter and/or sheath, facilitating advancing the contact assembly 810 through the vasculature of the patient. The second end-portion 850C of each arm of the connecting component 850 can be configured to converge towards a region opposite to the tip and/or head 851 to facilitate coupling the plurality of arms to the conduit 860. In some embodiments, the second end-portion 850C of each arm of the connecting component 850 can be aligned parallel to the second end-portions 850C of all other arms of the connecting component 850 and at a very close distance when they are not coupled to the conduit 860, as shown in FIGS. 14A and 14B. When the connecting component 850 is coupled to the conduit 860, the second end-portions 852 of the arms can be disposed and/or placed inside an interior volume of the conduit 860 and secured via a coupling mechanism such as an interference coupling. In that way, the second end-portions 852 of the arms of the connecting component 850 can assume a non-planar orientation in which the second-end-portions 850C are oriented around a perimeter of the interior volume of the conduit 860.

FIGS. 14A and 14B show the connecting component 850 includes a V-shape feature 852. The V-shape feature 852 facilitates folding and/or contracting the connecting component 850 such that the contact assembly 810 (with the conductive elements 820, the supporting tubes 830 A-D and the conductive wire 840) can assume the compressed configuration. Similarly, the V-shape figured 852 facilitates expanding the connecting component 850 such that the contact assembly 810 assumes the expanded configuration. In some embodiments, the connecting component 850 can include one or more sections having a reduced cross-sectional area that facilitates folding/unfolding the connecting component 850 such that the contact assembly 810 can be transitioned between the compressed configuration and the expanded configuration. For example, as shown in FIG. 14A, the connecting component 850 can include one or more regions 850D having a smaller cross-sectional area (and/or smaller width, height, thickness, diameter, etc.) to facilitate folding and/or contracting the contacting component 850.

Although the contact assembly 810 is shown and described as having a V-shape feature 852, in some embodiments the V-shape feature can be replaced with one or more lateral or angled (e.g., angled relative to a longitudinal axis defined by the conduit and/or the mid-section 850B) segments or supports, disposed between the first end portion 850A of each arm, and the tip 851. In this manner, the arms do not need to be formed separately and then coupled together at the tip or otherwise. Further, in this manner, the one or more segments or supports can help support termination of the supporting tubes 830 and/or the conductive wire 840.

The apparatus 800 can include a plurality of conductive elements 820. For example, as illustrated in FIG. 13, in some implementations the apparatus 800 can include 20 conductive elements 820. Alternatively, in some implementations the apparatus 800 can include any suitable number of conductive elements. FIG. 13 shows the apparatus 800 includes a conductive wire 840 disposed and/or mounted on a central arm of the connecting component 850. The conductive wire 840 can be a structure that defines an interior volume (e.g., a lumen) that can be used to accommodate the central (or other) arm of the connecting component 850. In some implementations, the conductive wire 840 can be made of spiral cut stainless steel configured to possess sufficient flexibility to facilitate transitioning the contact assembly 810 from an expanded configuration to a compressed configuration, as disclosed above. In some embodiments, the conductive wire 840 can be and/or include a Nitinol hypo tube. In some embodiments, the conductive wire 840 can be made of electrically conductive materials such as copper, gold, platinum, steel, nickel, titanium, or a combination thereof. In some embodiments the conductive wire 840 can be made a metal coated with platinum or including a radiopaque wound platinum wire to facilitate imaging the conductive wire 840 with a fluoroscope, and/or any suitable imaging modality. FIG. 13 shows the apparatus 800 can also include two conductive elements 820A (e.g., ring electrodes) disposed on an outer surface of the conduit 860. In some implementations, the conductive elements 820A can be used as reference and/or ground electrodes to facilitate taking measurements from the conductive elements 820 disposed on the supporting tubes 830 and the skeleton-like connecting component 850. FIG. 13 shows in some implementations, a portion of the conductive wire 840 can be covered and/or surrounded by an insulating layer 841, particularly near a proximal end-portion of the conductive wire 840 that is coupled to the conduit 860. In some embodiments, the insulating layer 841 can be tucked away inside the conduit 860 to prevent the conductive wire 840 to short circuit with the leads of adjacent conductive elements 820 or their leads disposed within the conduit 860.

FIGS. 15A and 15B show a perspective and top view, respectively, of an intracardiac apparatus 900 for mapping the anatomy of a patient, according to an embodiment of the present disclosure. The intracardiac apparatus 900, which can also be referred to as the apparatus 900, or the catheter 900, can be the same or similar in form and/or function to the apparatus 100, 200, 300, and 800, described above with reference to FIGS. 1-5. 13, 14A, and 14B. For example, as described above with reference to the apparatus 100, the apparatus 900 can be configured to be introduced via an incision into the body of a patient and be used to measure electrical data that can be used to diagnose tissue of the patient and/or to generate a map (e.g., mapping) the anatomy of the patient. The apparatus 900 includes a contact assembly 910 disposed on a distal end of a conduit 960. The contact assembly 910 includes a plurality of conductive element(s) 920, a plurality of supporting tube(s) 930, and a connecting component 950. In some implementations, portions and/or aspects of the apparatus 900 can be similar to and/or substantially the same as portions and/or aspects of the apparatus 100, 200, 300, and/or 800 described above with reference to FIGS. 1-5. 13, 14A, and 14B. Accordingly, such similar portions and/or aspects may not be described in further detail herein.

FIG. 15A shows the connecting component 950 is a skeleton-like monolithic structure including a plurality of segments, sections, spines, tines, arms, or the like that provide mechanical support to the conductive elements 920, and the supporting tubes 930. More specifically, the connecting component 950 includes a tip and/or head 951, and five different segments, sections, spines, tines, arms, or the like, that stem from a V-shape feature 952 coupled to the tip and/or head 951 of the connecting component 950. The five arms of the connecting component 950 are sized and configured to provide mechanical support to the supporting tubes 930A, 930B, 930C, 930D, and 930E (e.g., the supporting tubes 930A-E). The supporting tubes 930A-E can be coupled and/or mounted on the arms of the connecting component 950 by introducing each arm within an interior volume and/or lumen defined by the supporting tubes 930 A-E. Said in other words, each one of the supporting tubes 930 A-E can be moved along or slid over the arms of the connecting component 950. FIG. 15A shows each supporting tube 930 includes a plurality of conductive elements 930 disposed on a surface of the supporting tube 930. As disclosed above, in some embodiments each conductive element 920 can include a lead wire coupled to and/or attached to the conductive element 920 to connect electrically the conductive element 920 with a connector similar to and/or the same as the connectors shown in FIGS. 7A, 7B, and 11. The lead wires of the conductive elements 920 shown in FIG. 15A can be accommodated within an interior volume of the supporting tubes 930 that is not occupied by the arms of the connecting component 950. Said in another way, each lead wire can be placed within an annular region and/or volume defined as the interior volume of a supporting tube 930 that is not occupied by an arm of the connecting component 950. In some embodiments, each lead wire can be coated or covered with an insulating material to prevent short-circuiting one or more conductive elements 920.

The apparatus 900 can include any suitable number of conductive elements 920. For example, in some implementations, as shown in FIG. 15A, the apparatus 900 can include 25 conductive elements 920. In other implementations, as shown in FIG. 15B, the apparatus 900 can include 27 conductive elements 920. In some implementations the conductive elements 920, or at least a portion thereof, can be disposed on the supporting tubes 930 according to a predetermined pattern and/or arrangement. For example, in some implementations the conductive elements 920 (or a portion thereof) can be disposed on the supporting tubes 930 forming a two-dimensional flat array and/or matrix in which the conductive elements 920 are organized in columns and rows at specific and/or predetermined distances from each other. In some implementations, as shown in FIG. 15B, the conductive elements 920 can be disposed on each supporting tube 930 spaced out a predetermined distance “b”, measured as a center-to-center distance between two consecutive conductive element 920. The conductive elements 920 can also be disposed spaced out a predetermined center-to-center distance “a” from adjacent supporting tubes 930, as shown in FIG. 15B. In some implementations, the distance “a” can be different from the distance “b.” For example, in some implementations, the conductive elements 920 (or a portion thereof) can be disposed on the supporting tubes 930 forming a two-dimensional flat array and/or matrix in which the conductive elements 920 are organized in columns and rows defined by distance and/or spacings defined by a ratio of the distance “b” to the distance “a” (e.g., a “b/a” ratio, also referred to herein as an “interspacing to lateral spacing” ratio). In some implementations the “b/a” ratio can be at least about 0.50, at least about 0.55, at least about 0.60, at least about 0.65, at least about 0.70, at least about 0.75, at least about 0.80, at least about 0.85, at least about 0.90, at least about 0.95, at least about 1.0, at least about 1.1, at least about 1.2, at least about 1.3, at least about 1.4, or at least about 1.5, inclusive of all values and rages therebetween. In some implementations the “b/a” ratio can be no more than about 1.5, no more than about 1.4, no more than about 1.3, no more than about 1.2, no more than about 1.1, no more than about 1.0, no more than about 0.9, no more than about 0.8, no more than about 0.7, no more than about 0.6, or no more than about 0.5, inclusive of all values and ranges therebetween.

In some implementations, the conductive elements 920 (or a portion thereof) can be disposed on the supporting tubes 930 forming a two-dimensional flat array and/or matrix in which (1) conductive elements 920 mounted on the most external and/or outer supporting tubes 930 (e.g., supporting tubes 930A and 930B shown in FIG. 15A) are placed at a first center-to-center distance (e.g., a distance “a1”) from conductive elements 920 mounted on the adjacent supporting tubes 930C and 930D, and (2) conductive elements 920 mounted on the central supporting tube 930E are placed at a second center-to-center distance (e.g., a distance “a2”) from the adjacent supporting tubes 930C and 930C, with the second center-to-center distance “a2” being different from the first center-to-center distance “a1.”

FIG. 15B shows in some implementations the contact assembly 910 can also include two or more additional conductive elements 920 (e.g., conductive elements 920x and 920y) disposed near and/or adjacent to the tip and/or head 951. The conductive elements 920x and 920y can be disposed near the tip and/or head 951 (or between the tip and/or head 951 and the remaining conduct elements 920) to enhance the signals produced and/or measured by contact assembly 910, since during operation of the apparatus 900, in some implementations, contact with tissue is greater towards the distal end of the apparatus 900, such that, e.g., a majority of the conductive elements 920 in direct contact with tissue of a user and/or patient may be located in the vicinity of the distal terminal end and/or tip 951. Consequently, the use of additional conductive elements 920 disposed near the distal terminal end and/or tip 951 (as shown with conductive elements 920x and 920y in FIG. 15B) facilitates measuring signals of the tissue with high fidelity without capturing signals from other areas of the field such as the blood pool within a heart chamber or the opposite side of the heart.

FIG. 15A shows in some implementations the apparatus 900 can also include two or more conductive elements 920A disposed on the conduit 960 and configured to be used as reference and/or ground electrodes that facilitate taking measurements from the conductive elements 920 disposed on the supporting tubes 930 and the skeleton-like connecting component 950. The conductive elements 920A can be any suitable optical and/or electromagnetic (EM) tracking electrode and/or sensor, including, for example, NDI's Aurora®, 3D Guidance® EM tracking sensors. The conductive elements 920A can provide additional degrees of freedom for mapping the apparatus 900. For example, in some embodiments each conductive element 920A can impart 5-degrees of freedom. Optionally, in some embodiments the apparatus 900 can include two conductive element 920A collectively imparting 6-degrees of freedom. In some embodiments, the conduit 960 can be configured to flow irrigation solutions, as disclosed above with reference to the conduit 660 in FIG. 8.

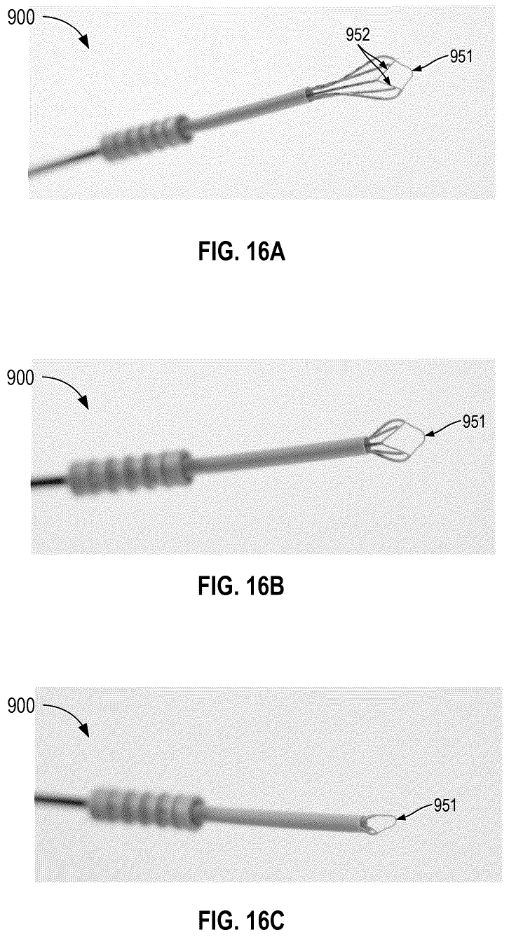

FIGS. 16A, 16B, and 16C show a perspective view of the apparatus 900 during transitioning of the contact assembly 910 from the expanded configuration to the compressed configuration. As shown in FIGS. 16A and 16B, the connecting component 950 of the apparatus 900 includes a V-shaped feature 952 that facilitates folding and/or contracting the connecting component 950 such that the contact assembly 910 (with the conductive elements 920, the supporting tubes 930 A-E) can assume the compressed configuration and fit within a delivery catheter and/or sheath to advance the contact assembly 910 through the vasculature of the heart of a patient. It is worth noting that the-shaped feature 952 can deform and/or contract to facilitate collapsing the contact assembly 910 in a reproducible manner. Similarly, the V-shaped feature 952 can expand to facilitate expanding the contact assembly 910 such that each supporting tube 930 is oriented according to the in-plane and/or planar orientation) disclosed above.

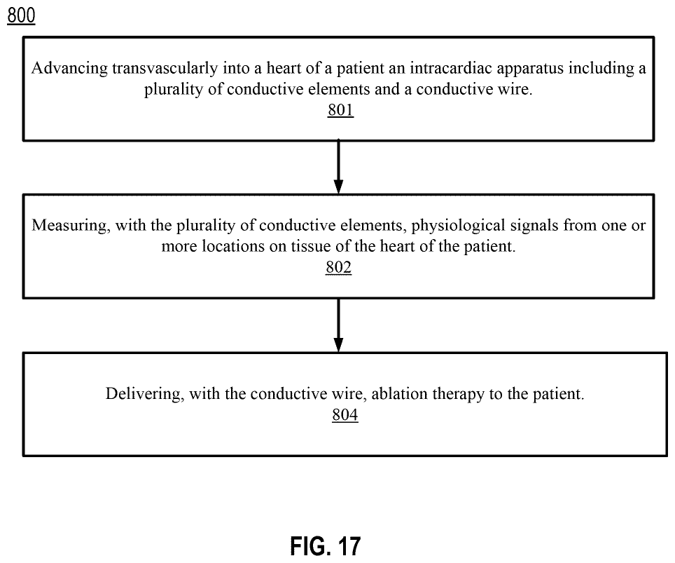

FIG. 17 illustrates an example method 800 for measuring physiological signals and delivering ablation therapy to tissue of a heart of a patient, according to an embodiment of the present disclosure. The method 800 includes at 801, advancing transvascularly (e.g., via a delivery catheter) into a heart of a patient, an intracardiac apparatus including a plurality of conductive elements and a conductive wire. In some implementations, the plurality of conductive elements can be ring electrodes configured to measure signals such as voltage, current, impedance, and the like. In some implementations, the conductive elements can be microelectrodes having a nominal length of at least about 0.5 mm to no more than about 1.0 mm, and/or a nominal outer diameter of at least about 0.6 mm to no more than about 0.85 mm. Each conductive element from the plurality of conductive elements can be electrically insulated from all the other conductive elements. The conductive wire can be a wire of metal configured to establish a potential and/or voltage with respect to one or more conductive element from the plurality of conductive elements. The potential and/or voltage established by the conductive wire can be any potential and/or voltage suitable for delivering ablation therapy, including, for example, irreversible electroporation ablation (also referred to as pulse field ablation, PFA), to the patient. In some implementations, the potential and/or voltage established and/or produced by the conductive wire can be at least about 5000 V to no more than about 15,000V. In some implementations, the intracardiac apparatus can be configured to fit within a delivery catheter or a sheath such that the intracardiac apparatus can be received at the heart of the patient. For example, in some implementations the intracardiac apparatus can be configured to fit within a delivery catheter or sheath having an outer diameter of no more than about 8.5 Fr (e.g., 8.5 Fr, 8 Fr, 7.5 Fr, etc.). In some implementations, the conductive elements and the conductive wire can be electrically and operably coupled to a cardiac mapping system, a pulse field ablation (PFA) generator, or any other suitable device capable of generating 3D maps of the anatomy of a patient and/or delivering energy therapies. In some implementations, the intracardiac apparatus can be substantially similar to and/or the same as the apparatus 100, 200, 300, 800, and/or 900 described above with reference to FIGS. 1-5, and 13-16.

At 802 the method 800 includes measuring, with the plurality of conductive elements, physiological signals from one or more locations on tissue of the heart of the patient. In some implementations, the plurality of conductive elements can be configured to be disposed in direct contact and/or in close proximity of tissue of the heart of the patient to measure physiologic parameters of the tissue of the patient such as voltage, current, impedance, and/or timing of cardiac depolarization. In some implementations, the conductive elements can be configured to contact tissue of the patient to facilitate high fidelity signal acquisition, e.g., to precisely identify cellular ectopic foci. In some implementations, the voltages measured by the conductive elements can be used to assess the health of the tissue of the heart of the patient. Low voltage measurements can be used to identify areas of scar tissue while higher voltage measurements can be used to identify areas of healthy tissue that is depolarizing with each heartbeat. In some implementations, the electrical data measured by the conductive elements can be associated with three dimensional locations in space that facilitate the reconstruction of 3D physiologic maps of the heart of the patient.

At 803, the method 800 includes delivering, with the conductive wire, ablation therapy to the patient. As described above, the conductive wire can be configured to establish a potential and/or voltage with respect to one or more conductive element from the plurality of conductive elements, which can be used to deliver ablation therapy to tissue of the heart of the patient. In some implementations, the conductive wires can be configured to deliver these voltages for a short period of time according to predetermined frequencies and/or voltage intensities.

While various embodiments described herein include delivering electroporation ablation therapy and/or a conductor configured to deliver electroporation therapy, it should be understood that any of the embodiments described herein can be configured to delivery electroporation ablation therapy using any suitable polarity. In some implementations, for example, unipolar electroporation ablation may be delivered from the conductor to a patch (e.g., a ground patch) or the like, on or near the patient. In some implementations, for example, unipolar electroporation ablation may be delivered from one or more conductive elements to a patch (e.g., a ground patch) or the like, on or near the patient. Further, in some implementations, a mixture of unipolar and bipolar electroporation ablation therapy may be provided. For example, using a conductor (e.g., tubular electrode), one or more conductive elements (e.g., ring electrodes), and a ground patch on the patient, both unipolar and bipolar electroporation ablation may be used.

While various embodiments described herein including delivering electroporation ablation therapy across a components on an apparatus (e.g., a contact assembly) and/or from the apparatus to a patch, embodiments described herein can also be configured to or can include delivering electroporation ablation therapy via a separate device or catheter disposed within the patient's body. For example, bipolar electroporation ablation energy may be delivered from one or more conductive elements and/or a conductor (e.g., tubular electrode) to a separate device or catheter inserted within the patient. Further to this example, in some instances, a contact assembly can be placed on the outside of a patient's heart (e.g., epicardial access through a subxiphoid approach) and ablate from inside or across the heart with a catheter (e.g., a linear catheter) disposed within the heart to create lesions (e.g., relatively deep lesions, such as, for example, over about 2 cm thick) in the ventricle(s).

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” Any ranges cited herein are inclusive.

The terms “substantially,” “approximately,” and “about” used throughout this Specification and the claims generally mean plus or minus 10% of the value stated, e.g., about 100 would include 90 to 110.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, the term “about” and “approximately” generally mean plus or minus 10% of the value slated, e.g., about 250 μm would include 225 μm to 275 μm, about 1,000 μm would include 900 μm to 1,100 μm. As used herein in the specification and in the claims, the term “aspect ratio” can be defined as a ratio of an in-plane lateral dimension to the thickness of the final product.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

1. An apparatus, comprising:
a conduit having a proximal end and a distal end, the conduit defining a longitudinal axis extending from the proximal end to the distal end; and
a contact assembly extending from the distal end of the conduit, the contact assembly including:
a monolithic connecting component configured to provide structural support to the contact assembly, including a tube support section and a lateral support section disposed between the tube support section and a distal tip of the contact assembly;
a plurality of tubes disposed on the tube support section of the connecting component; and
a plurality of conductive elements disposed on the plurality of tubes and configured to measure physiological signals associated with the patient.

2. The apparatus of claim 1, further comprising an elongated conductor coupled to the connecting component and configured to deliver electroporation ablation therapy to a patient, wherein each conductive element from the plurality of conductive elements is electrically isolated from the elongated conductor.

3. The apparatus of claim 1, wherein the plurality of conductive elements is configured to measure physiological signals from various locations on tissue of a heart of the patient.

4. The apparatus of claim 1, wherein the conductive elements are ring electrodes, each ring electrode having a nominal length of at least about 0.5 mm to no more than about 1.0 mm.

5. The apparatus of claim 4, wherein each ring electrode has a nominal outer diameter of at least about 0.6 mm to no more than about 0.85 mm.

6. The apparatus of claim 1, wherein one or more of the conductive elements from the plurality of conductive elements are configured to measure a voltage with respect to a ground electrode, the ground electrode being spaced from the plurality of conductive elements, the voltage being associated with depolarization of the tissue of the heart during a heartbeat.

7. The apparatus of claim 1, wherein the plurality of conductive elements is configured to measure a plurality of voltages to collectively construct a three-dimensional map of electrical propagation through the heart during a heartbeat.

8. The apparatus of claim 1, wherein the lateral support section is structurally weaker than the tube support section such that the connecting component can be flexed concavely or convexly.

9. The apparatus of claim 1, wherein the connecting component is configured to place the contact assembly in an expanded configuration in which the plurality of supporting tubes is oriented along a plane and each supporting tube from the plurality of supporting tubes is disposed parallel to all the other supporting tubes from the plurality of supporting tubes such that the contact assembly can lay flat at a close proximity of or flush to intracardiac tissue of a patient.

10. The apparatus of claim 9, wherein the connecting component includes an insulating material.

11. The apparatus of claim 10, wherein the insulating material is nylon.

12. The apparatus of claim 9, wherein the connecting component includes a conductive material.

13. The apparatus of claim 12, wherein the conductive material is nitinol.

14. The apparatus of claim 9, wherein the lateral support section includes a V-shape with arms and a vertex, with the arms extending distally from the vertex.

15. The apparatus of claim 8, wherein the contact assembly is configured to transition from the expanded configuration to a compressed configuration in which the plurality of supporting tubes are compressed towards each other such that the contact assembly has an outer diameter substantially similar to an inner diameter of the conduit.

16. The apparatus of claim 8, wherein the outer diameter of the conduit is no more than about 8.5 Fr.

17. The apparatus of claim 2, wherein the elongated conductor is configured to establish a potential with respect to one or more conductive elements from the plurality of conducting elements, the potential capable of delivering energy to cause electroporation of cardiac cells included in tissue of a heart of a patient.

18. The apparatus of claim 17, wherein the potential is at least about 3,000 volts to no more than about 15,000 volts.

19. The apparatus of claims 2, wherein the elongated conductor is made of nitinol.

20. The apparatus of claim 3, wherein the elongated conductor has a nominal diameter of at least about 0.2 millimeters and no more than about 1 mm, and has a nominal length of at least 2 mm and no more than about 20 mm.

21. The apparatus of claim 1, wherein the distal tip is convex and atraumatic, and the plurality of tubes terminate proximal to the lateral support section.

22. An apparatus, comprising:
a conduit having a proximal end and a distal end, the conduit defining a longitudinal axis extending from the proximal end to the distal end; and
a contact assembly extending from the distal end of the conduit, the contact assembly including:
a connecting component configured to provide structural support to the contact assembly, including a tube support section and a lateral support section disposed between the tube support section and a distal tip of the contact assembly;
a plurality of tubes disposed on the tube support section of the connecting component;
a plurality of conductive elements disposed on the plurality of tubes and configured to measure physiological signals associated with a patient; and
an elongated conductor coupled to the connecting component and configured to deliver electroporation ablation therapy to the patient, wherein each conductive element from the plurality of conductive elements is electrically isolated from the elongated conductor.

23. The apparatus of claim 22, wherein the distal tip includes at least one of a lubricant coating or a biocompatible plastic coating.

24. The apparatus of claim 22, wherein the plurality of conductive elements is configured to measure the physiological signals to collectively construct a three-dimensional map of the heart of the user.

25. The apparatus of claim 22, wherein the plurality of supporting tubes include wound coils, the wound coils configured to interact magnetically with an electromagnetic localization sensor to determine a location of the plurality of supporting tubes with respect to the heart of the user.

26. The apparatus of claim 22, wherein the conductive elements are ring electrodes, each ring electrode having a nominal length of at least about 0.5 mm to no more than about 1.0 mm.

27. The apparatus of claim 22, wherein the lateral support section is structurally weaker than the tube support section such that the connecting component can be flexed concavely or convexly.

28. The apparatus of claim 22, wherein the connecting component is configured to place the contact assembly in an expanded configuration in which the plurality of supporting tubes is oriented along a plane and each supporting tube from the plurality of supporting tubes is disposed parallel to all the other supporting tubes from the plurality of supporting tubes such that the contact assembly can lay flat at a close proximity of or flush to intracardiac tissue of a patient.

29. The apparatus of claim 22, wherein the elongated conductor is a Nitinol tube disposed about the connecting component.

30. The apparatus of claim 22, wherein the lateral support section includes a V-shape with arms and a vertex, with the arms extending distally from the vertex.
